(19) United States
(12) Patent Application Publication
(10) Pub. No.: US 2023/0115463 A1
(43) Pub. Date: Apr. 13, 2023

(54) CANNABIDIOL-CONTAINING COMPOSITIONS AND USES THEREOF

(71) Applicant: Ramot at Tel-Aviv University Ltd., Tel-Aviv (IL)

(72) Inventors: Daniel OFFEN, Tel-Aviv (IL); Felix BENNINGER, Tel-Aviv (IL); Nataly YOM TOV, Tel-Aviv (IL)

(73) Assignee: Ramot at Tel-Aviv University Ltd., Tel-Aviv (IL)

(21) Appl. No.: 17/959,327

(22) Filed: Oct. 4, 2022

Related U.S. Application Data
(63) Continuation of application No. PCT/IL2021/050406, filed on Apr. 7, 2021.
(60) Provisional application No. 63/006,099, filed on Apr. 7, 2020.

(51) Int. Cl. A61K 47/54; A61K 9/127; A61K 35/28; A61K 31/05; A61P 31/00; A61P 25/28; A61P 25/08

(52) U.S. Cl. CPC A61K 47/548; A61K 9/127; A61K 35/28; A61K 31/05; A61P 31/00; A61P 25/28; A61P 25/08

(57) ABSTRACT

Compositions comprising a cell-derived particle associated with Cannabidiol (CBD), and uses thereof in treating medical conditions that can benefit from Cannabidiol (CBD) are provided.

### RELATED APPLICATIONS

This application is a Continuation of PCT Patent Application No. PCT/IL2021/050406 having International filing date of Apr. 7, 2021, which claims the benefit of priority under 35 USC § 119(e) of U.S. Provisional Patent Application No. 63/006,099 filed on Apr. 7, 2020. The contents of the above applications are all incorporated by reference as if fully set forth herein in their entirety.

U.S. Provisional Patent Application No. 63/006,099 was also co-filed with U.S. Provisional Patent Application No. 63/171,686, having Attorney Docket No. 86779, the contents of which are also incorporated by reference as if fully set forth herein in its entirety.

### FIELD AND BACKGROUND OF THE INVENTION

The present invention, in some embodiments thereof, relates to therapy, and, more particularly, but not exclusively, to cannabidiol-containing compositions and uses thereof in treating diseases that can benefit from Cannabidiol.

The cannabis plant (Marijuana) has many naturally occurring substances that are of great interest in the fields of science and medicine. Isolated compounds from the cannabis plant are collectively referred to herein and in the art as cannabinoids or phytocannabinoids, and include, inter 25 alia, ^{Δ}9-tetrahydrocannabinol (THC), cannabidiol (CBD), cannabichromene (CBC), cannabigerol (CBG), cannabinol (CBN), cannabidivarin (CBDV). While THC has psychoactive effects. CBD does not.

Cannabinoids can be isolated by extraction or cold pressing from cannabis plants. Plants in the cannabis genus from which cannabinoids can be extracted include Cannabis sativa, Cannabis ruderalis, and Cannabis indica. Cannabinoids can also be synthetically prepared (synthesized).

Preparations of Marijuana and pure phytocannabinoids [e.g. Cannabidiol (CBD)] were shown to have anticonvulsive, sedative, hypnotic, anti-psychotic, anti-oxidant, anti-inflammatory and neuroprotective effects and were suggested as treatments for varying medical conditions disorders including chronic pain, epilepsy, cancer, neurodegenerative disorders and cognitive disorders such as Alzheimer's disease and dementia behavioral disorders. Autism spectrum disorders (ASD), bipolar disorder, schizophrenia and anxiety disorders [See, e.g., Watt G. and Karl T. (2017) Front. Pharmacol. 8:20; Poleg et al. (2019) Progress in Neuropsychopharmacology & Biological Psychiatry 89: 90-96].

Delivering CBD into various bodily targets, however, is still challenging.

Mesenchymal stem cells (MSC) have been reported to show therapeutic effects in many animal models of inflammation and injury. More than thousand clinical studies are currently listed in the clinicaltrial(dot)gov website. Several endowments have already been approved for use by the FDA.

The usefulness of the cells has been tested and demonstrated in a number of biological systems, indicating the potential efficacy in a number of diseases where there are joint spreads, such as inflammation and neurodegenerative disorders. See, for example. Volkman and Offen, Stem Cells, 2017 November; 35(11):2321.

Extracellular vesicles (EVs) are particles with a lipid bilayer that are naturally released from a cell, but which cannot replicate. EVs may be released from the surface of cells, in which case they are referred to as ectosomes, microvesicles or microparticles; or in endosomal compartments which release the EVs when the endosomal compartment fuses with the cell surface, in which case they are referred to as exosomes. Exosomes are generally smaller (about 30 to 150 nm in diameter) than most other EVs, as their size is limited by the size of their endosomal compartment. EVs such as exosomes often comprise proteins and RNA (e.g., micro RNAs), and are hypothesized to play a natural role in cell-to-cell signaling.

Exosomes derived from mesenchymal stem cells (MSCs) exhibit effects similar to the MSCs, such as promotion of tissue damage repair, suppressing inflammatory responses, regulation of neurite outgrowth, promotion of angiogenesis, and immune system modulation [Guy & Offen, Biomolecules 2020, 10:1320]. For example, intranasal administration of exosomes secreted from MSCs was reported to ameliorate autistic-like behaviors in a mouse model [Perets et al., Mol Autism 2018, 9:57] and to result in exosome accumulation in pathologically relevant brain regions in mouse models of stroke, autism, Parkinson's disease and Alzheimer's disease in a manner which was highly correlated to neuro-inflammation [Perets et al., Nano Lett 2019, 19:3422-3431].

Exosomes were initially thought to be a mechanism for removing unneeded membrane proteins from reticulocytes but current studies have shown they are used for cell-to-cell communication by carrying information from one cell to another. Several studies have reported that MSC-derived exosomes have functions similar to those of MSCs, such as repairing tissue damage, suppressing inflammatory responses, and modulating the immune system [Yu et. al. (2014) Int. J. Mol. Sci. 15(3): 4142-4157].

Exosomes are easily traceable and can be targeted to specific areas, which makes it easier to follow their mechanism of action compared to cells (Valadi et al., 2007, Nature Cell Biology, Vol. 9, pp. 654-659). Furthermore, their very small size (˜00 nm) and their capacity to penetrate the blood brain barrier (BBB) and migrate to lesion sites enables penetration into the brain.

Exosomes derived from several cell types including MSCs and neural stem cells have been suggested for the treatment of several disorders including inflammatory disorders, cancer, autoimmune disorders, lung diseases, nerve injury and neurodegenerative disorders e.g. Alzheimer's disease and Parkinson's disease [See, e.g., Zhuang et. al. (2011) Mol. Ther. 19(10): 1769-79; Yu et al. (2014) Int. J. Mol. Sci. 15(3): 4142-4157; Sun G, et al. (2018) Mater. Sci. Eng. C Mater. Biol. Appl. 89:194-204; Liu et al. (2020) Expert Opin. Biol. Ther. 20(2): 125-140; U.S. Patent Application Publication No. 2015/0190430; International Patent Application Publication No. WO 2013/150303].

Besides their natural biological properties, extracellular vesicles (EVs) such as exosomes have been considered as promising carriers for drug loading and delivery, due to their ability to cross various biological/physical barriers such as the blood-brain barrier (BBB), stability and non-immunogenicity (which protects their cargo), non-toxicity relative to synthetic nanoparticles, and ability to target specific sites. Exosomes have been loaded with nucleic acids via co-incubation of exosomes and nucleic acids, which suffers from low loading efficiency; by transfection of exosome-producing cells, which is costly, time-consuming and hard to quantify; and by electroporation, which affects exosome integrity [Fu et al., NanoImpact 2020, 20:100261; Jafari et al., BioDrugs 2020, 34:567-586].

Modification of EVs to incorporate various types of pharmacological agents have been explored in numerous contexts. For instance. WO 2013/084000 describes the use of exosomes for intracellular delivery of biotherapeutics. WO 2010/119256, describes delivery of exogenous genetic material using exosomes.

Exosomes have been loaded with nucleic acids via co-incubation of exosomes and nucleic acids, which suffers from low loading efficiency; by transfection of exosome-producing cells, which is costly, time-consuming and hard to quantify; and by electroporation, which affects exosome integrity [Fu et al., NanoImpact 2020, 20:100261; Jafari et al., BioDrugs 2020, 34:567-586].

WO 2011/097480 describes a typical approach to loading of EVs with organic small molecule compounds. WO 2011/097480 describes a very facile method wherein e.g. the phytochemical agents curcumin and resveratrol are loaded into EVs using a simple co-incubation step during which purified EVs and free drug (e.g. curcumin) are allowed to incubate together in phosphate buffered saline (PBS) at room temperature, relying on diffusion of the drug into the EV. Although highly convenient and straightforward, this conventional approach to loading simple pharmacological agents into EVs is not particularly efficient, results in significant waste of the pharmacological agent, and is also very difficult to control. Also, it suffers from lack of general applicability, as some pharmacological agents will not load into EVs in high quantities only using co-incubation.

WO 2015/120150 describes loading of tumor-derived EVs with various types of anticancer drugs, covering both small molecular agents and large biopharmaceuticals.

WO 2018/011153 describes the use of cell penetrating peptides (CPPs) to carry agents, such as siRNA, mRNA and peptides, into EVs.

Exo-Fect™ transfection kits are a commercial product utilizing CPP technology to transfer siRNA or mRNA into EVs such as exosomes.

Some of the present inventors have recently reported that MSC derived exosomes loaded with phosphatase and tensin homologue (PTEN) small interfering RNA could attenuate the expression of PTEN in the injured spinal cord region following intranasal administrations [Guo et al., ACS Nano, 2019, 2019 Sep. 24; 13(9):10015-10028, reported].

Additional background art includes K Cheung et al. (2019) Int. J. Mol. Sci. 20(23): 6079, and WO 2019/186558.

### SUMMARY OF THE INVENTION

According to an aspect of some embodiments of the present invention there is provided a method of treating a medical condition (e.g., a disease or disorder) that can benefit from Cannabidiol (CBD) in a subject in need thereof, the method comprising administering to the subject a therapeutically effective amount of a composition comprising a stem or progenitor cell-derived particle associated with (e.g., encapsulating) CBD, thereby treating the disease in the subject.

According to an aspect of some embodiments of the present invention there is provided a composition comprising a stem or progenitor cell-derived particle associated with (e.g., encapsulating) Cannabidiol (CBD), for use in treating a medical condition (e.g., a disease or disorder) that can benefit from CBD in a subject in need thereof.

According to an aspect of some embodiments of the present invention there is provided a method of treating a medical condition (e.g., a disease or disorder) that can benefit from Cannabidiol (CBD) in a subject in need thereof, wherein the disease is not epilepsy, the method comprising administering to the subject a therapeutically effective amount of a composition comprising a cell-derived particle associated with (e.g., encapsulating) CBD, thereby treating the disease in the subject.

According to an aspect of some embodiments of the present invention there is provided a composition comprising a cell-derived particle associated with (e.g., encapsulating) Cannabidiol (CBD), for use in treating a medical condition (e.g., a disease or disorder) that can benefit from CBD, wherein the disease is not epilepsy, in a subject in need thereof.

According to some of any of the embodiments of the invention, the cell is a stem or progenitor cell.

According to some of any of the embodiments of the invention, the stem or progenitor cell is selected from the group consisting of a mesenchymal stem cell (MSC), neuronal stem cells (NSC), neuronal crest cell (NCC).

According to some of any of the embodiments of the invention, the cell is a mesenchymal stem cell (MSC).

According to some of any of the embodiments of the invention, the cell-derived particle is selected from the group consisting of an exosome, ARRM, microvesicle, exomere, membrane particle, membrane vesicle and extosome.

According to some of any of the embodiments of the invention, the cell-derived particle is an exosome.

According to some of any of the embodiments of the invention, the cell-derived particle is a mesenchymal stem cell (MSC)-derived exosome.

According to some of any of the embodiments of the invention, the medical condition is selected from the group consisting of epilepsy, neurodegenerative disease, nerve injury, stroke, inflammation and infectious disease.

According to some of any of the embodiments of the invention, the disease is epilepsy.

According to some of any of the embodiments of the invention, the disease is selected from the group consisting of neurodegenerative disease, nerve injury, stroke, inflammation and infectious disease.

According to some embodiments of the invention, the disease is Alzheimer's disease.

According to some embodiments of the invention, the disease is an infectious disease.

According to some embodiments of the invention, the infectious disease is a virus-induced pneumonia.

According to some embodiments of the invention, the infectious disease is a Corona virus infection.

According to some embodiments of the invention, the Coronavirus is SAR-CoV-2. Middle East respiratory syndrome Coronavirus or severe acute respiratory syndrome Coronavirus.

According to some of any of the embodiments described herein, the medical condition is treatable by modulating an activity of a CB1 receptor.

According to some of any of the embodiments of the invention, the composition is administered intranasally.

According to some of any of the embodiments of the invention, the composition is administered by inhalation.

According to some of any of the embodiments of the present invention, the CBD is a modified CBD, which comprises at least one substituent or moiety attached to a position of the CBD.

According to some of any of the embodiments of the present invention, the substituent or moiety is attached to position 6 and/or 5″ of the CBD.

According to some of any of the embodiments of the present invention, the modified CBD comprises a phospholipid moiety conjugated therewith, directly or via a linking moiety.

According to some of any of the embodiments of the present invention, the substituent or moiety is capable of facilitating an association between the CBD and the particle.

According to an aspect of some embodiments of the present invention there is provided a composition comprising a cell-derived particle and a cannabidiol associated with the particle.

According to some of any of the embodiments of the present invention, the cell is a stem or progenitor cell.

According to some of any of the embodiments of the present invention, the stem or progenitor cell is selected from the group consisting of a mesenchymal stem cell (MSC), neuronal stem cells (NSC), neuronal crest cell (NCC).

According to some of any of the embodiments of the present invention, the cell is a mesenchymal stem cell (MSC).

According to some of any of the embodiments of the present invention, the cell-derived particle is selected from the group consisting of an exosome, ARRM, microvesicle, exomere, membrane particle, membrane vesicle and extosome.

According to some of any of the embodiments of the present invention, the cell-derived particle is an exosome.

According to some of any of the embodiments of the present invention, the cell-derived particle is a mesenchymal stem cell (MSC)-derived exosome.

According to some of any of the embodiments of the present invention, the CBD is a modified CBD, which comprises at least one substituent or moiety attached to a position of the CBD.

According to some of any of the embodiments of the present invention, the substituent or moiety is attached to position 6 and/or 5″ of the CBD.

According to some of any of the embodiments of the present invention, the modified CBD comprises a phospholipid moiety conjugated therewith, directly or via a linking moiety.

According to some of any of the embodiments of the present invention, the substituent or moiety is capable of facilitating an association between the CBD and the particle.

According to some of any of the embodiments of the present invention, the composition is for use in the treatment of a medical condition that can benefit from CBD.

According to some of any of the embodiments of the present invention, the medical condition is treatable by modulating an activity of a CB1 receptor.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B present the chemical structure of cannabidiol. 1A) and the data obtained in an Auto-Dock vina docking analysis of CBD into a CB1 receptor (CB1 structure obtained from Hua. Tian, et al. “Crystal structure of the human cannabinoid receptor CB1.” Cell 167.3 (2016): 750-762.) (FIG. 1B).

FIGS. 2A-B present cartoon (FIG. 2A) and surface (FIG. 2B) structures of CB1 receptor (cyan) with CBD (green) inside the active site as predicted using AutoDock vina.

FIG. 3 presents a structure showing a bilayer phospholipid membrane of an exosome (structure obtained from Chung et al. PloS one 14.7 (2019): e0220025) and a phospholipid-CBD conjugate (green) anchored therewithin, as predicted using AutoDock vina.

### DESCRIPTION OF SPECIFIC EMBODIMENTS OF THE INVENTION

The present invention, in some embodiments thereof, relates to therapy, and, more particularly, but not exclusively, to cannabidiol-containing compositions and uses thereof in treating diseases that can benefit from Cannabidiol.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

While, as described hereinabove, both exosomes and non-psychoactive phytocannabinoids such as CBD exhibits various therapeutic effects, the present inventors have conceived encapsulating CBD or CBD-containing materials in exosomes or other cell-derived particles (e.g., extracellular vesicles) so as to use such compositions in a synergic manner in the treatment of diseases that can benefit from CBD.

Embodiments of the present invention therefore relate to compositions comprising a cell-derived particle (e.g., an extracellular vesicle) encapsulating CBD and to uses thereof.

According to an aspect of some embodiments of the present invention, there is provided a method of treating a disease that can benefit from CBD in a subject in need thereof, the method comprising administering to the subject a therapeutically effective amount of a composition comprising a cell-derived particle (e.g., an extracellular vesicle) encapsulating CBD, thereby treating the disease in the subject.

According to an aspect of some embodiments of the present invention, there is provided a composition comprising a cell-derived particle (e.g., an extracellular vesicle) encapsulating CBD, for use in treating a disease that can benefit from CBD in a subject in need thereof.

According to an aspect of some embodiments of the present invention, there is provided a use of a composition comprising a cell-derived particle (e.g., an extracellular vesicle) encapsulating CBD, in the manufacture of a medicament for use in treating a disease that can benefit from CBD in a subject in need thereof.

According to an aspect of some embodiments of the present invention, there is provided a method of treating a disease that can benefit from CBD in a subject in need thereof, the method comprising administering to the subject a therapeutically effective amount of a composition comprising a stem or progenitor cell-derived particle encapsulating CBD, thereby treating the disease in the subject.

According to an aspect of some embodiments of the present invention, there is provided a composition comprising a stein or progenitor cell-derived particle encapsulating CBD, for use in treating a disease that can benefit from CBD in a subject in need thereof.

According to an aspect of some embodiments of the present invention, there is provided a use of a composition comprising a stem or progenitor cell-derived particle encapsulating CBD, in the manufacture of a medicament for use in treating a disease that can benefit from CBD in a subject in need thereof.

According to an additional or an alternative aspect of the present invention, there is provided a method of treating a disease that can benefit from CBD in a subject in need thereof, wherein the disease is not epilepsy, the method comprising administering to the subject a therapeutically effective amount of a composition comprising a cell-derived particle encapsulating CBD, thereby treating the disease in the subject.

According to an additional or an alternative aspect of the present invention, there is provided a composition comprising a cell-derived particle encapsulating CBD, for use in treating a disease that can benefit from CBD, wherein the disease is not epilepsy, in a subject in need thereof.

Particle:

The term “particle” as used herein refers to a cell-derived particle having an internal space surrounded by a lipid bilayer (e.g., cell membrane). It will be appreciated that particle is not an intact cell and does not have the ability to replicate.

The particle may generally features a shape of a vesicle or a flattened sphere.

According to specific embodiments, the particle generally features a shape of a vesicle.

The particle may have a size greater than 2 nm. The particle may have a size greater than 5 nm. 10 nm, 20 nm, 30 nm, 40 nm or 50 nm. The particle may have a size greater than 100 nm, such as greater than 150 nm. The particle may have a size of substantially 200 nm or greater.

The particle or particles may have a range of sizes, such as from about 2 nm to about 20 nm, from about 2 nm to about 50 nm, from about 2 nm to about 100 nm, from about 2 nm to about 150 nm, or from about 2 nm to about 200 nm, or higher, for example, from about 2 nm to about 250 nm, or from about 2 nm to about 300 nm, or from about 2 nm to about 500 nm, or from about 2 nm to about 1000 nm, including any intermediate values and subranges therebetween. The particle or particles may have a size that ranges from about 30 to about 1000 nm. The particle or particles may have a size that ranges from about 20 nm to about 50 nm, or from about 20 nm to about 100 nm, or from about 20 nm to about 150 nm, or from about 20 nm to about 200 nm, including any intermediate values and subranges therebetween. The particle or particles may have a size that ranges from about 50 nm to about 100 nm, or from about 50 nm to about 150 nm, or from about 50 nm to about 200 nm, including any intermediate values and subranges therebetween. The particle or particles may have a size that ranges from about 100 nm to about 150 nm, or from about 100 nm to about 200 nm, including any intermediate values and subranges therebetween. The particle or particles may have a size that ranges from about 150 nm to about 200 nm, including any intermediate values and subranges therebetween.

The size may be determined by various means. In principle, the size may be determined by size exclusion methods, for example, by size fractionation and filtration through a membrane with the relevant size cut-off. The particle size may then be determined by tracking segregation of component proteins with SDS-PAGE or by a biological assay. Whenever a “size” of a particle is described herein, it refers to at least one dimension of the particle, for example, diameter, and, it refers to an average size of a plurality of particles.

The size of the particle may alternatively by reflected as its hydrodynamic radius. The hydrodynamic radius of the particle may be below 100 nm. It may be between about 30 nm and about 70 nm. The hydrodynamic radius may be between about 40 nm and about 60 nm, such as between about 45 nm and about 55 nm. The hydrodynamic radius may be about 50 nm.

The hydrodynamic radius of the particle may be determined by any suitable means, for example, laser diffraction or dynamic light scattering.

The particles may have a density of about 1.13-1.19 grams/ml and may float on sucrose gradients. The particles may be enriched in cholesterol and sphingomyelin, and lipid raft markers such as GM1, GM3, flotillin and the src protein kinase Lyn.

As used herein, the term “cell-derived” refers to a particle produced within, by or from a biological cell. The particle may be derivable from the cell by any of several means, for example by secretion, budding or dispersal from the cell. For example, the particle may be produced, exuded, emitted or shed from the cell. Where the cell is in cell culture, the particle may be secreted into the cell culture medium.

The particles may comprise one or more macromolecules present in the cell or the culture medium, including nucleic acids, proteins, carbohydrates, lipids, small molecules and/or combinations thereof. Such macromolecules are typically characteristic or specific to the cell or the medium. In a particular embodiment, the particle may comprise miRNA.

For example, the particle may comprise 10%; or more, 20% or more, 30% or more, 40% or more, 50% or more. 60% or more or 70% or more of these macromolecules, e.g., proteins and/or polynucleotides. The particle may comprise substantially about 75% of these macromolecules, e.g., proteins and/or polynucleotides. The proteins may be defined by reference to a list of proteins or gene products of a list of genes.

The particle may be isolated or isolatable from a cell or a culture medium. The particle may be responsible for at least an activity of the cell it is derived from or the culture medium. For example, the particle may be a substitute (or biological substitute) for the cell or the culture medium.

The particle preferably has at least one property of the cell it is derived from. The particle may have a biological property, such as a biological activity. The particle may have any of the biological activities of the cell it is derived from. The particle may for example have a therapeutic or restorative activity of the cell it is derived from.

Non-limiting examples of particles that can be used according to some exemplary embodiments of the present invention include an exosome. ARRM, microvesicle, exomere, membrane particle, membrane vesicle and extosome.

According to some embodiments, the cell-derived particle is also referred to herein as an extracellular vesicle.

According to specific embodiments, the particle is an exosome.

As used herein, the term “exosome” refers to an extracellular vesicle that is released from a cell upon fusion of a multivesicular body (MV B) with the plasma membrane.

The exosome may (a) have a size of between 50 nm and 100 nm as determined by electron microscopy; (b) comprise a complex of molecular weight higher than 100 kDa, comprising proteins of molecular weight lower than 100 kDa; (c) comprise a complex of molecular weight higher than 300 kDa, comprising proteins of molecular weight lower than 300 kDa; (d) comprise a complex of molecular weight higher than 1000 kDa; (e) have a size of between about 2 nm and about 200 nm, as determined by filtration against a 0.2 pM filter and concentration against a membrane with a molecular weight cut-off of 10 kDa; or (f) have a hydrodynamic radius of below 100 nm, as determined by laser diffraction or dynamic light scattering.

The cells from which the particles of some embodiments of the invention may be derived from may be of any source and or any tissue origin. Non-limiting examples of tissues include neural tissue, kidney tissue, lung tissue, bone marrow, cord blood, adipose tissue, dental pulps.

According to some of any of the embodiments described herein, the cell is a mammalian cell. According to specific embodiments, the cell is a human cell.

The cell may be a primary cell or an immortalized cell line.

According to some of any of the embodiments described herein, the cell is a primary cell. Non-limiting examples of cells that are usable in the context of the present embodiments include neuronal cells, kidney cells, hematopoietic cells, adipocytes.

The cell may be a fully differentiated cell or a stem or progenitor cell.

According to some of any of the embodiments described herein, the cell is a stem or progenitor cell.

As used herein, the phrase “stem or progenitor cell” refers to a cell capable of undergoing mitotic division and differentiating into other cell types having a particular, specialized function (e.g., fully differentiated cells), and includes e.g. a totipotent cell, a pluripotent cell or a multipotent cell and may refer to a cell committed to a specific lineage.

Non-limiting Examples of stem or progenitor cells from which the particles may be derived from include, embryonic stem cells, induced pluripotent stem cells (iPS), adult stem or progenitor cells, bone marrow-derived stem or progenitor cells, hematopoietic progenitor cells, mesenchymal stem cells (MSCs), neuronal stem cells (NSCs), neural crest cell (NCC), oral mucosa stem cells.

According to some of any of the embodiments described herein, the stem or progenitor cell is selected from the group consisting of a mesenchymal stem cell (MSC), neuronal stem cells (NSC) and neuronal crest cell (NCC).

The cell may be a primary cell or an immortalized cell line (e.g., HEK-293, NIH3T3).

Methods of isolating, purifying and expanding stem or progenitor cells are well known to the skilled in the art and are further described hereinbelow.

The phrase “embryonic stem cells (ESC)” refers to embryonic cells which are capable of differentiating into cells of all three embryonic germ layers (i.e., endoderm, ectoderm and mesoderm), or remaining in an undifferentiated state. The phrase “embryonic stem cells” may comprise cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO2006/040763), embryonic germ (EG) cells which are obtained from the genital tissue of a fetus any time during gestation, preferably before 10 weeks of gestation, and cells originating from an unfertilized ova which are stimulated by parthenogenesis (parthenotes).

The embryonic stem cells of some embodiments of the invention can be obtained using well-known cell-culture methods. For example, human embryonic stem cells can be isolated from human blastocysts. Human blastocysts are typically obtained from human in vivo preimplantation embryos or from in vitro fertilized (IVF) embryos. Alternatively, a single cell human embryo can be expanded to the blastocyst stage. For the isolation of human ES cells the zona pellucida is removed from the blastocyst and the inner cell mass (ICM) is isolated by immunosurgery, in which the trophectoderm cells are lysed and removed from the intact TCM by gentle pipetting. The ICM is then plated in a tissue culture flask containing the appropriate medium which enables its outgrowth. Following 9 to 15 days, the ICM derived outgrowth is dissociated into clumps either by a mechanical dissociation or by an enzymatic degradation and the cells are then re-plated on a fresh tissue culture medium. Colonies demonstrating undifferentiated morphology are individually selected by micropipette, mechanically dissociated into clumps, and re-plated. Resulting ES cells are then routinely split every 4-7 days. For further details on methods of preparation human ES cells see Thomson et al., [U.S. Pat. No. 5,843,780; Science 282: 1145, 1998; Curr. Top. Dev. Biol. 38: 133, 1998; Proc. Natl. Acad. Sci. USA 92: 7844, 1995]; Bongso et al., [Hum Reprod 4: 706, 1989]; and Gardner et al., [Fertil. Steril. 69: 84, 1998].

It will be appreciated that commercially available stem cells can also be used according to some embodiments of the invention. Human ES cells can be purchased from the NIH human embryonic stem cells registry [Hypertext Transfer Protocol://grants (dot) nih (dot) gov/stem_cells/registry/current (dot) htm]. Non-limiting examples of commercially available embryonic stem cell lines are BG01, BG02, BG03, BG04, CY12, CY30, CY92, CY10, TE03, TE32, CHB-4, CHB-5, CHB-6, CHB-8, CHB-9, CHB-10, CHB-11, CHB-12, HUES 1, HUES 2, HUES 3, HUES 4, HUES 5, HUES 6, HUES 7, HUES 8, HUES 9, HUES 10, HUES 11, HUES 12, HUES 13, HUES 14, HUES 15, HUES 16, HUES 17, HUES 18, HUES 19, HUES 20, HUES 21, HUES 22, HUES 23, HUES 24, HUES 25, HUES 26, HUES 27, HUES 28, CyT49, RUES3, WAO1, UCSF4, NYUES1, NYUES2, NYUES3, NYUES4, NYUES5, NYUES6, NYUES7, UCLA 1, UCLA 2, UCLA 3, WA077 (H7), WA09 (H9), WA13 (H13), WA14 (H14), HUES 62, HUES 63, HUES 64, CT1, CT2, CT3, CT4, MA135, Eneavour-2, WIBR1, WIBR2, WIBR3, WIBR4, WIBR5, WIBR6, HUES 45, Shef 3, Shef 6, BJNhem19, BJNhem20, SA001, SA001.

In addition, ES cells can be obtained from non-human species as well, including mouse (Mills and Bradley, 2001), golden hamster [Doctschman et al., 1988, Dev Biol. 127: 224-7], rat [Iannaccone et al., 1994. Dev Biol. 163: 288-92] rabbit [Giles et al. 1993, Mol Reprod Dev. 36: 130-8; Graves & Moreadith. 1993, Mol Reprod Dev. 1993, 36: 424-33], several domestic animal species [Notarianni et al., 1991, J Reprod Fertil Suppl. 43: 255-60; Wheeler 1994, Reprod Fertil Dev. 6: 563-8; Mitalipova et al., 2001, Cloning. 3: 59-67] and non-human primate species (Rhesus monkey and marmoset) [Thomson et al., 1995, Proc Natl Acad Sci USA. 92: 7844-8; Thomson et al., 1996, Biol Reprod. 55: 254-9].

Extended blastocyst cells (EBCs) can be obtained from a blastocyst of at least nine days post fertilization at a stage prior to gastrulation. Prior to culturing the blastocyst, the zona pellucida is digested [for example by Tyrode's acidic solution (Sigma Aldrich, St Louis, Mo., USA)] so as to expose the inner cell mass. The blastocysts are then cultured as whole embryos for at least nine and no more than fourteen days post fertilization (i.e., prior to the gastrulation event) in vitro using standard embryonic stem cell culturing methods.

Another method for preparing ES cells is described in Chung et al., Cell Stem Cell, Volume 2, Issue 2, 113-117, 7 Feb. 2008. This method comprises removing a single cell from an embryo during an in vitro fertilization process. The embryo is not destroyed in this process.

EG cells are prepared from the primordial germ cells obtained from fetuses of about 8-11 weeks of gestation (in the case of a human fetus) using laboratory techniques known to anyone skilled in the arts. The genital ridges are dissociated and cut into small chunks which are thereafter disaggregated into cells by mechanical dissociation. The EG cells are then grown in tissue culture flasks with the appropriate medium. The cells are cultured with daily replacement of medium until a cell morphology consistent with EG cells is observed, typically after 7-30 days or 1-4 passages. For additional details on methods of preparation human EG cells see Shamblott et al., [Proc. Natl. Acad. Sci. USA 95: 13726, 1998] and U.S. Pat. No. 6,090,622.

Embryonic stem cells (e.g., human ESCs) originating from an unfertilized ova stimulated by parthenogenesis (parthenotes) are known in the art (e.g., Zhenyu Lu et al., 2010. J. Assist Reprod. Genet. 27:285-291; “Derivation and long-term culture of human parthenogenetic embryonic stem cells using human foreskin feeders”, which is fully incorporated herein by reference). Parthenogenesis refers to the initiation of cell division by activation of ova in the absence of sperm cells, for example using electrical or chemical stimulation. The activated ovum (parthenote) is capable of developing into a primitive embryonic structure (called a blastocyst) but cannot develop to term as the cells are pluripotent, meaning that they cannot develop the necessary extra-embryonic tissues (such as amniotic fluid) needed for a viable human foetus.

According to specific embodiments, the cell is not an embryonic stem cell.

As use herein the phrase “induced pluripotent stem cells (iPS; embryonic-like stem cells)”, refers to cells obtained by de-differentiation of adult somatic cells which are endowed with pluripotency (i.e., being capable of differentiating into the three embryonic germ cell layers, i.e., endoderm, ectoderm and mesoderm). According to some embodiments of the invention, such cells are obtained from a differentiated tissue (e.g., a somatic tissue such as skin) and undergo de-differentiation by genetic manipulation which re-program the cell to acquire embryonic stem cells characteristics.

Induced pluripotent stem cells (iPS) (embryonic-like stem cells) can be generated from somatic cells by genetic manipulation of somatic cells, e.g., by retroviral transduction of somatic cells such as fibroblasts, hepatocytes, gastric epithelial cells with transcription factors such as Oct-3/4, Sox2, c-Myc, and KLF4 [Yamanaka S, Cell Stem Cell. 2007, 1(1):39-49; Aoi T, et al., Generation of Pluripotent Stem Cells from Adult Mouse Liver and Stomach Cells. Science. 2008 Feb. 14. (Epub ahead of print); I H Park, Zhao R. West J A, et al. Reprogramming of human somatic cells to pluripotency with defined factors. Nature 2008; 451:141-146; K Takahashi. Tanabe K, Ohnuki M, et al. Induction of pluripotent stem cells from adult human fibroblasts by defined factors. Cell 2007; 131:861-872]. Other embryonic-like stem cells can be generated by nuclear transfer to oocytes, fusion with embryonic stem cells or nuclear transfer into zygotes if the recipient cells are arrested in mitosis.

The phrase “adult stem or progenitor cells” (also called “tissue stem cells” or a stem cell from a somatic tissue) refers to any stem or progenitor cell derived from a somatic tissue [of either a postnatal or prenatal animal (especially the human)]. The adult stem or progenitor cell is generally thought to be a multipotent stem cell, capable of differentiation into multiple cell types. Adult stem or progenitor cells can be derived from any adult, neonatal or fetal tissue such as adipose tissue, skin, kidney, liver, prostate, pancreas, intestine, bone marrow and placenta.

Adult tissue stem or progenitor cells can be isolated using various methods known in the art such as those disclosed by Alison, M. R. [J Pathol. 2003 200(5): 547-50], Cai, J, et al., [Blood Cells Mol Dis. 2003 31(1): 18-27]. Collins. A. T, et al., [J Cell Sci. 2001; 114(Pt 21): 3865-72]. Potten, C. S. and Morris, R. J. [Epithelial stem cells in vivo. 1988. J. Cell Sci. Suppl. 10, 45-62], Dominici, M et al., [J. Biol. Regul. Homeost. Agents. 2001, 15: 28-37], Caplan and Haynesworth [U.S. Pat. No. 5,486,359] Jones E. A, et al., [Arthritis Rheum. 2002, 46(12): 3349-60]. Generally, isolation of adult tissue stem or progenitor cells is based on the discrete location (or niche) of each cell type included in the adult tissue, i.e., the stem cells, the transit amplifying cells and the terminally differentiated cells [Potten. C. S. and Morris. R. J. (1988). Epithelial stem cells in vivo. J. Cell Sci. Suppl. 10, 45-62].

The phrase “neural stem cells (NSCs)”, refers to cells capable of differentiating into neurons, astrocytes, oligodendrocytes and/or glial cells, or remaining in an undifferentiated state.

Neural stem cells can be isolated using various methods known in the arts such as those disclosed by Svendsen et al. (1999) Brain Pathol. 9(3): 499-513. Rietze and Reynolds (2006) Methods Enzymol. 419:3-23; and “Handbook of Stem Cells” edit by Robert Lanze, Elsevier Academic Press. 2004.

The phrase “hematopoietic stein or progenitor cells” includes stem or progenitor cells obtained from blood or bone marrow tissue of an individual at any age or from cord blood of a newborn individual.

Hematopoietic stem or progenitor cells can be isolated using various methods known in the arts such as those disclosed by “Handbook of Stem Cells” edit by Robert Lanze, Elsevier Academic Press. 2004. Chapter 54, pp 609-614, isolation and characterization of hematopoietic stem cells, by Gerald J Spangrude and William B Stayton.

According to specific embodiments, the stem or progenitor cells are BM-derived stem cells including hematopoietic, stromal or mesenchymal stem cells (Dominici. M et al., 2001. Bone marrow mesenchymal cells: biological properties and clinical applications. J. Biol. Regul. Homeost. Agents. 15: 28-37). BM-derived stem cells may be obtained from iliac crest, femora, tibiae, spine, rib or other medullar spaces.

According to specific embodiments, the cell is a mesenchymal stem cell (MSC).

According to a specific embodiment, the particle is an MSC-derived exosome.

As used herein, the term “mesenchymal stem cells (MSCs)” refers to multipotent stromal cells that can differentiate into a variety of cell types, including: osteoblasts (bone cells), chondrocytes (cartilage cells), myocytes (muscle cells) and adipocytes (fat cells).

In their pluripotent state, mesenchymal stem cells typically express the following markers: CD105, CD166, CD29, CD90, and CD73, and do not express CD34, CD45, and CD133.

Mesenchymal stem cells may be isolated from a variety of tissues including but not limited to bone marrow, adipose tissue, dental pulp, oral mucosa, peripheral blood and amniotic fluid.

Methods of isolating, purifying and expanding mesenchymal stem cells (MSCs) are known in the arts and include, for example, those disclosed by Caplan and Haynesworth in U.S. Pat. No. 5,486,359 and Jones E. A, et al., 2002, Isolation and characterization of bone marrow multipotential mesenchymal progenitor cells, Arthritis Rheum. 46(12): 3349-60.

Preferably, mesenchymal stem cell cultures are generated by diluting BM aspirates (usually 20 ml) with equal volumes of Hank's balanced salt solution (HBSS; GIBCO Laboratories. Grand Island, N.Y. USA) and layering the diluted cells over about 10 ml of a Ficoll column (Ficoll-Paque; Pharmacia, Piscataway, N.J., USA). Following 30 minutes of centrifugation at 2,500×g, the mononuclear cell layer is removed from the interface and suspended in HBSS. Cells are then centrifuged at 1.500×g for 15 minutes and resuspended in a complete medium (MEM, a medium without deoxyribonucleotides or ribonucleotides; GIBCO); 20% fetal calf serum (FCS) derived from a lot selected for rapid growth of MSCs (Atlanta Biologicals, Norcross. Ga.); 100 units/ml penicillin (GIBCO). 100 μg/ml streptomycin (GIBCO); and 2 mM L-glutamine (GIBCO). Resuspended cells are plated in about 25 ml of medium in a 10 cm culture dish (Corning Glass Works. Corning. N.Y.) and incubated at 37° C. with 5% humidified CO₂. Following 24 hours in culture, nonadherent cells are discarded, and the adherent cells are thoroughly washed twice with phosphate buffered saline (PBS). The medium is replaced with a fresh complete medium every 3 or 4 days for about 14 days. Adherent cells are then harvested with 0.25% trypsin and 1 mM EDTA (Trypsin/EDTA, GIBCO) for 5 min at 37° C., replated in a 6-cm plate and cultured for another 14 days. Cells are then trypsinized and counted using a cell counting device such as for example, a hemocytometer (Hausser Scientific, Horsham. Pa.). Cultured cells are recovered by centrifugation and resuspended with 5% DMSO and 30% FCS at a concentration of 1 to 2×10⁶ cells per ml. Aliquots of about 1 ml each are slowly frozen and stored in liquid nitrogen.

To expand the mesenchymal stem cell fraction, frozen cells are thawed at 37° C. diluted with a complete medium and recovered by centrifugation to remove the DMSO. Cells are resuspended in a complete medium and plated at a concentration of about 5,000 cells/cm². Following 24 hours in culture, nonadherent cells are removed and the adherent cells are harvested using Trypsin/EDTA, dissociated by passage through a narrowed Pasteur pipette, and preferably replated at a density of about 1.5 to about 3.0 cells/cm². Under these conditions, MSC cultures can grow for about 50 population doublings and be expanded for about 2000 fold [Colter D C., et al. Rapid expansion of recycling stem cells in cultures of plastic-adherent cells from human bone marrow. Proc Natl Acad Sci USA. 97: 3213-3218, 2000].

MSC cultures utilized by some embodiments of the invention include three groups of cells which are defined by their morphological features: small and agranular cells (referred to as RS-1, hereinbelow), small and granular cells (referred to as RS-2, hereinbelow) and large and moderately granular cells (referred to as mature MSCs, hereinbelow). The presence and concentration of such cells in culture can be assayed by identifying a presence or absence of various cell surface markers, by using, for example, immunofluorescence, in situ hybridization, and activity assays.

The particle may be produced or isolated in a number of ways. Such a method may comprise isolating the particle from a cell e.g. MSC. Such a method may comprise isolating the particle from a culture medium e.g. MSC conditioned medium (MSC-CM). Thus, according to specific embodiments, the composition comprising the particles is cell-free, i.e. does not comprise a detectable amount of cells.

The particle may be isolated for example by being separated from non-associated components based on any property of the particle. For example, the particle may be isolated based on molecular weight, size, shape, composition or biological activity.

The conditioned medium may be filtered or concentrated or both during, prior to or subsequent to separation. For example, it may be filtered through a membrane, for example one with a size or molecular weight cut-off. It may be subject to tangential force filtration or ultrafiltration.

For example, filtration with a membrane of a suitable molecular weight or size cutoff, as described in the Assays for Molecular Weight elsewhere in this document, may be used.

The conditioned medium, optionally filtered or concentrated or both, may be subject to further separation means, such as column chromatography. For example, high performance liquid chromatography (HPLC) with various columns may be used. The columns may be size exclusion columns or binding columns.

One or more properties or biological activities of the particle may be used to track its activity during fractionation of the culture medium. As an example, light scattering, refractive index, dynamic light scattering or UV-visible detectors may be used to follow the particles. For example, a therapeutic activity such as cardioprotective activity may be used to track the activity during fractionation.

The following paragraphs provide a non-limiting example of how a MSC-derived particle such as an exosome may be obtained.

A MSC-derived exosome may be produced by culturing MSCs in a medium to condition it. The medium may comprise DMEM. The DMEM may be such that it does not comprise phenol red. The medium may be supplemented with insulin, transferrin, or selenoprotein (ITS), or any combination thereof. It may comprise FGF2. It may comprise PDGF AB. The concentration of FGF2 may be about 5 ng/ml FGF2. The concentration of PDGF AB may be about 5 ng/ml. The medium may comprise glutamine-penicillin-streptomycin or -mercaptoethanol, or any combination thereof.

The cells may be cultured for about 1, 2, 3, 4, 5, 6, 7, 8, 9, 10 days or more, for example 3 days. The conditioned medium may be obtained by separating the cells from the medium. The conditioned medium may be centrifuged, for example at 500 g. It may be concentrated by filtration through a membrane. The membrane may comprise a >1000 kDa membrane. The conditioned medium may be concentrated about 50 times or more.

The conditioned medium may be subjected to liquid chromatography such as HPLC. The conditioned medium may be separated by size exclusion. Any size exclusion matrix such as Sepharose may be used. As an example, a TSK Guard column SWXL. 6×40 mm or a TSK gel G4000 SWXL. 7.8×300 mm may be employed. The eluent buffer may comprise any physiological medium such as saline. It may comprise 20 mM phosphate buffer with 150 mM of NaCl at pH 7.2. The chromatography system may be equilibrated at a flow rate of 0.5 ml/min. The elution mode may be isocratic. UV absorbance at 220 nm may be used to track the progress of elution. Fractions may be examined for dynamic light scattering (DLS) using a quasi-elastic light scattering (QELS) detector.

Fractions which are found to exhibit dynamic light scattering may be retained. For example, a fraction which is produced by the general method as described above, and which elutes with a retention time of 11-13 minutes, such as 12 minutes, is found to exhibit dynamic light scattering. The rₙ of particles in this peak is about 45-55 nm. Such fractions comprise MSCs-derived exosomes.

According to some of any of the embodiments described herein, the composition comprises a plurality of cell-derived particles, wherein at least a portion of the particles are cell-derived particles encapsulating CBD, as described herein in any of the respective embodiments.

By “at least a portion” it is meant that at least 10%, or at least 20%, or at least 30%, or at least 40%, or at least 50%, or at least 60%, preferably at least 70%, or at least 80%, or at least 90%, or even about 100%, of the particles are cell-derived particles encapsulating CBD, as described herein in any of the respective embodiments. The particles in the plurality of particles can be substantially identical to one another.

The particles described herein in any of the respective embodiments and any combination thereof encapsulate cannabidiol (CBD), as defined and described herein in any of the respective embodiments and any combination thereof.

As used herein the term “encapsulate” or “encapsulating” has the meaning of supplemented or filled with the CBD and includes entrapped within the interior of particle, exposed or present at the surface of the particle (either inner and/or outer surface), embedded in the particle lipid bilayer and/or entrapped with the liquid phage of the particle.

The term “encapsulate” or “encapsulating” or any grammatical diversion thereof, is also referred to herein interchangeably as “associated with” or “in association with”.

By “associated with” and grammatical diversions thereof it is meant that the CBD and the particle are in association with one another, whereby the association can be a chemical interaction (e.g., a chemical bond such as a covalent bond, an electrostatic bond, a hydrogen bond) or a physical interaction (e.g., encapsulation, entrapment, deposition, absorption, etc.).

More specifically, by “associated therewith” it is meant that the CBD is in chemical or physical interaction with the particle (at least a portion of the particle), whereby in some embodiments, this interaction is not a result of a mere mutual presence in the same environment, mixture, medium or matrix.

Thus, for example, the CBD can be associated with the particle, by interacting with functional groups present in the particle via, e.g., covalent bonds, electrostatic interactions, hydrogen bonding, van der Waals interactions, donor-acceptor interactions, aromatic (e.g., π-π interactions), or cation-n interactions. These interactions lead to the chemical association of the CBD to the particle.

Alternatively, the CBD can be associated with the particle by physical association such as surface adsorption, encapsulation, entrapment, entanglement and the likes.

It is contemplated that encapsulation or association of the CBD in the particle is performed in a manner that does not impede the therapeutic activity of the CBD.

Encapsulation or association of the CBD in the particle may be performed by incubating the particle with the CBD under conditions (e.g. time, temperature, pH, medium etc.) sufficient to permit encapsulation.

For example, incubation of about 1 hour or less is sufficient to permit encapsulation or association of the CBD in the particle. According to specific embodiments, the incubation period is less than 5 minutes, at least 5 minutes, at least 10 minutes, at least 20 minutes, at least 30 minutes. According to specific embodiments, the incubation period is for at least 1 hour, at least 2 hours, at least 3 hours, at least 4 hours, at least 5 hours, at least 10 hours, at least 12 hours, at least 24 hours.

According to some embodiments, encapsulation is effected at around 37° C.

According to some embodiments, encapsulation is effected at around 4° C.

According to some embodiments, encapsulation is effected at room temperature.

According to some of any of the embodiments described herein, the particles encapsulating the CBD are further purified or isolated using e.g. ultracentrifugation.

The Cannabidiol:

Cannabidiol (CBD) (CAS No. 13956-29-1) as used herein encompasses native CBD (i.e. originating from the Cannabis plant), synthetically prepared CBD, and synthetic analogs or derivatives thereof, including cannabidiol-containing conjugates as described in further detail hereinbelow. According to specific embodiments, any CBD analog may be used in accordance with specific embodiments of the present teachings as long as it exhibits the therapeutic activity of CBD, for example, binding or interacting with a CB receptor (e.g., CB1).

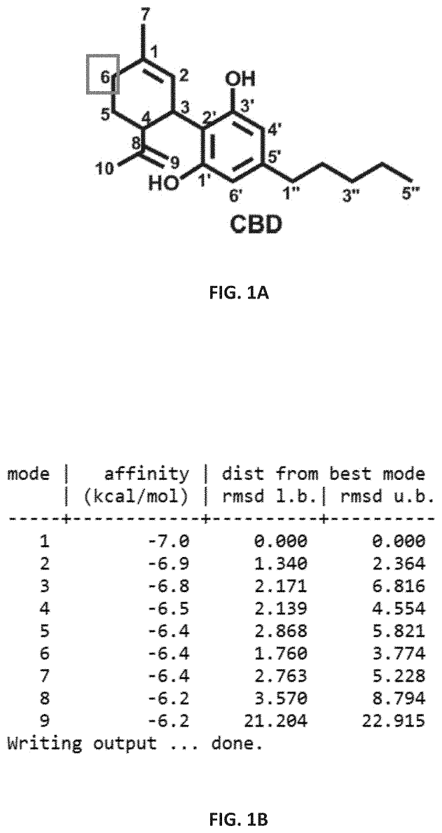

In some embodiments, the CBD analog or derivative is also referred to herein as “modified CBD” and describes compounds featuring a CBD skeleton (see, FIG. 1A) in which one or more positions are substituted or attached to another moiety, while maintaining the biological activity of CBD as described herein.

Exemplary CBD analogs include, but are not limited to (−)-DMH-CBD-11-oic acid, HU-308 (commercially available e.g. from Tocris Bioscience, 3088). 0-1602 (commercially available e.g. from Tocris Bioscience 2797/10). DMH-CBD (commercially available e.g. from Tocris Bioscience, 1481) [as discussed in detail in Burstein S, Bioorg Med Chem. (2015) 23(7): 1377-85], Abn-CBD, HUF-101. CBDV, CBDM, CBND-C5. CBND-C3, 6-Hydroxy-CBD-triacetate or CBD-aldehyde-diacetate [as discussed in detail in An Overview on Medicinal Chemistry of Synthetic and Natural Derivatives of Cannabidiol. Frontiers in Pharmacology, June 2017|Volume 8|Article 422┘.

According to some embodiments, the CBD is or comprises naturally occurring CBD, purified, isolated or extracted from a cannabis plant.

Plant-derived, isolated or synthetic CBD can be commercially obtained from e.g. Restek catalog no. 34011.

According to some of any of the embodiments described herein, the CBD is or comprises a CBD-containing material, for example, a CBD-releasing material, which is capable of releasing CBD under physiological conditions.

For example, according to some of any of the embodiments described herein, the CBD is or comprises a conjugate of CBD and an additional moiety, covalently attached therebetween, and in some of these embodiments, the conjugate is cleavable under physiological conditions to thereby release CBD or a derivative or analog thereof, as defined and described herein.

As described in further detail in the Examples section that follows, the present inventors have investigated possible positions of the CBD molecule in order to determine which of these positions can be subject to substitution or conjugation while maintaining the CBD biological performance.

The present inventors uncovered that substitutions at positions 6 and/or 5″ are preferred. See, Example 4 in the Examples section that follows.

In some embodiments, the modified CBD is substituted at position 6 of the CBD skeleton (see, FIG. 1A), for example, by a moiety that facilitates its association with the particle.

In some embodiments, the modified CBD is substituted at position 5″ of the CBD skeleton (see, FIG. 1A), for example, by a moiety that facilitates its association with the particle.

According to some of any of the embodiments described herein, the CBD is a modified CBD, as described herein, and in some embodiments, it is a conjugate of CBD and a phospholipid moiety.

Exemplary CBD-phospholipid moiety according to these embodiments can be collectively represented by Formula I:

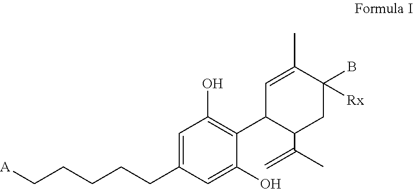

or a pharmaceutically acceptable salt thereof.

wherein:

A and B are each independently selected from hydrogen, alkyl, and L-P, wherein L is a linking moiety or absent and P is a phospholipid, provided that at least one of A and B is the L-P; and

Rx is hydrogen, or, when B is L-P, can be an alkyl, ether or amine linking group that forms a 5- or 6-membered ring with atoms of the L linking moiety, as described in further detail hereinbelow.

The conjugates described herein are of cannabidiol (see. FIG. 1A) conjugated to a phospholipid moiety, preferably via a linker, wherein the phospholipid moiety is attached either to position 5″ or to position 6 of the cannabidiol.

It is to be noted that the present embodiments encompass also conjugates of cannabidiol analogs or derivatives, in which one or more of positions 1, 2, 3, 4, 5, 6 (as long as not substituted by B), 9, 10, 4′, 6′, 1″, 2″, 3″, 4″ and 5″ (as long as not substituted by A) is substituted. Any of the substituents as described herein are contemplated.

Herein, the term “phospholipid” describes compounds that comprise a lipid moiety having a phosphate moiety attached thereto. Commonly available phospholipids are those belonging to the glycerophospholipid class, also known as phosphoglycerols or as mono- or di-acylglyceride phosphates. Other commonly available phospholipids include lipids having a (phosphorylated) sphingosine backbone, referred to as phosphosphingolipids (e.g., sphingomyelins).

Phosphoglycerols have a glycerolic backbone to which are attached one or two fatty acyl groups at positions sn-1 and/or sn-2, and one phosphate moiety at position sn-3.

Phosphosphingolipids have a sphingosine backbone which comprises one unsaturated fatty acyl, and to which are attached one fatty acyl via an amide bond and one phosphate moiety.

According to some of any of the embodiments of the invention, the phospholipid is phosphoglycerol.

The fatty acyl groups in a phospholipid as described herein may comprise saturated fatty acyl groups, monounsaturated fatty acyl groups (having a single unsaturated bond) and/or polyunsaturated fatty acyl groups (having two or more unsaturated bonds). In some embodiments, the unsaturated bonds are cis double bonds.

Examples of suitable saturated fatty acyl groups include, without limitation, lauroyl, myristoyl, palmitoyl and stearoyl.

Examples of suitable monounsaturated fatty acyl groups include, without limitation, oleoyl, palmitoleoyl, eicosenoyl, erucoyl, nervonoyl and vaccenoyl.

Examples of suitable polyunsaturated fatty acyl groups include, without limitation, linoleoyl, α-linolenoyl, γ-linolenoyl, dihomo-γ-linolenoyl, stearidonoyl, eicosatetraenoyl, eicosapentaenoyl, docosapentaenoyl, docosahexaenoyl, arachidonoyl and adrenoyl.

In some embodiments of any one of the embodiments described herein, the fatty acyl groups are selected from the group consisting of saturated and monounsaturated fatty acyl groups. In some embodiments, the fatty acyl groups are saturated fatty acyl groups.

In the conjugates as described herein, the phospholipid moiety is attached to the cannabidiol via the phosphate moiety.

In some embodiments, the phospholipid can be represented by the formula:

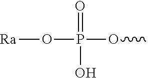

wherein Ra is a hydrocarbon of at least 4, or at least 6, or at least 8 carbon atoms in length (preferably Ra-O— representing a fatty acyl as described herein); and the curved line represents the attachment point to the linking moiety or the respective position of the CBD.

In some embodiments, the phospholipid can be represented by the formula:

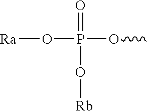

wherein Ra and Rb are each independently a hydrocarbon of at least 4, or at least 6, or at least 8 carbon atoms in length (preferably each of Ra-O— and Rb-O— independently represent a fatty acyl as described herein); and the curved line represents the attachment point to the linking moiety or the respective position of the CBD.

In some embodiments, the phospholipid is a phosphoglycerol as described herein and in the art and is represented by a formula:

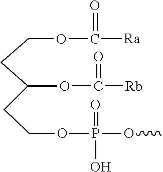

wherein Ra and Rb are each independently a hydrocarbon of at least 4, or at least 6, or at least 8 carbon atoms in length (preferably each of Ra-O— and Rb-O— independently represent a fatty acyl as described herein); and the curved line represents the attachment point to the linking moiety or the respective position of the CBD.

According to some of any of the embodiments described herein, the linking moiety is or comprises an alkylene chain, optionally interrupted by one or more heteroatoms.

The alkylene chain can be of 1, 2, 3, 4 or more carbon atoms in length, and is preferably from 1 to 4, or from 1 to 3, carbon atoms in length. The alkylene can be substituted or unsubstituted, as defined herein. In some embodiments, the alkylene is unsubstituted.

When the alkylene chain is interrupted by one or more heteroatoms, the heteroatoms can be oxygen, sulfur and/or nitrogen (e.g., as amine-linking group as defined herein).

According to some of any of the embodiments described herein, the linking moiety has a total of 1 to 20, or of 1 to 10, or of 1 to 4, atoms in length (including carbon atoms of the alkylene chain and one or more heteroatoms if such are present).

By “interrupted” in the context of heteroatoms in the linking moiety it is meant that a heteroatom is interposed between two carbon atoms of the alkylene chain, or is attached to one carbon atom of the alkylene chain and to the phosphate moiety and/or the respective position of the CBD.

According to some of any of the embodiments described herein, the linking moiety is an alkylene chain interrupted (as described herein) by one or more nitrogen atoms, and in some embodiments by one or more amine linking groups. Such linking moieties can be represented by the formula:

-(alkylene)n-(NR′)k-(alkylene)m-(NR″)j-

wherein the alkylene, R′ and R″ are as defined herein; and n, k, m and j are each independently 0 or 1, provided that at least one of n and m is 1 and at least one of k and j is 1. In some embodiments, R′ and R″, when present, are hydrogen.

The (alkylene)n and (alkylene)m in this formula form together the alkylene chain of the linking moiety.

Optionally, R′ and/or R″, as long as present, form together with two or more carbon atoms of the alkylene chain a nitrogen-containing heteroalicyclic moiety, such that the linking moiety can be or comprise one or more of the following non-limiting exemplary groups:

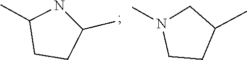

Further optionally, the nitrogen-containing heteroalicyclic moiety is formed with Rx (when B is L-P).

According to some of any of the embodiments described herein, the linking moiety is an alkylene chain interrupted by one or more oxygen atoms. Such linking moieties encompass linking moieties that comprise or consist of one or more ether-containing group(s).

By “ether-containing group” it is meant herein a moiety that comprises at least one alkylene-O-alkylene-group, for example a —(CR′R″)d-O—(CR′R″)e- group, wherein R′ and R″ are as defined herein and d and e are each independently 0 or an integer, such that d+e represented the number of carbon atoms in the respective portion of the linking moiety, or of the linking moiety as a whole.

In some embodiments, the ether-containing group is an oxygen-containing heteroalicyclic moiety, which comprises one or more (e.g., 2) oxygen atoms, for example, tetrahydrofuran, tetrahydropyran, dioxolanes (e.g., 1,3-dioxolane), dioxanes (e.g., 1,3-dioxane).

Exemplary such groups that can form a part or be the linking moiety include, but are not limited to:

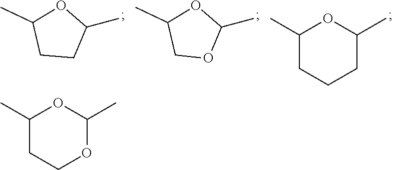

Further optionally, the nitrogen-containing heteroalicylic moiety is formed with Rx (when B is L-P).

According to some of any of the embodiments described herein, the linking moiety can be represented by Formula II:

-(CRuRy)f-X1-(CRwRz)g-X2-(CRqRt)h-

wherein:

Ru, Ry, Rw, Rz, Rq and Rt are each independently hydrogen, alkyl, amine, hydroxy, ether, or alkoxy;

X1 and X2 are each independently O, S, or NR′, or is absent; and

f, g, and h are each independent 0 or a positive integer, provided that at least one or f, g and h is a positive integer, and such that f+g+h represent the length of an alkylene chain of the linking moiety as described herein.

When f, g and/or h are 2 or higher, Ru and Ry, and/or Rw and Rz, and/or Rq and Rt, in each respective repeating group can be the same or different.

In some embodiments of Formula II, Ru, Ry, Rw, Rz, Rq and Rt is hydrogen, one of X1 and X2 is NR′ and the other is absent. In some of these embodiments, R′ is hydrogen. In some of these embodiments, X2 is absent, and g and h are each 0. In some of these embodiments, X2 is absent, g and h are each 0 and f is 2.

In some embodiments of Formula II, Ru, Ry, Rw. Rz, Rq and Rt is hydrogen, one of X1 and X2 is O and the other is absent.

In some embodiments of Formula II, f is a positive integer (e.g., 1 or 2), X1 is absent, g is 1, X2 is O, and h is a positive integer (e.g., 1 or 2). In some of these embodiments, Ru and Ry are each hydrogen. In some of these embodiments, one of Rw and Rz and one of Rq and Rt form together an oxygen-containing heterocylic ring as described herein. In some of these embodiments, one or more of Rw, Rz, Rq and Rt is an ether group, such that the oxygen-containing heterocylic ring includes 2 oxygen atoms (forming, for example, 1,3-dioxolane. 1,3-dioxane or 1,4-dioxane, each being optionally unsubstituted). In some of any of these embodiments, A is L-P.

In some embodiments of Formula II, f is a positive integer (e.g., 1 or 2). X1 is absent, g is 1. X2 is O. and h is 0, and B is L-P. In some of these embodiments, Ru and Ry are each hydrogen. In some of these embodiments, one of Rw and Rz and Rx form together an oxygen-containing heterocylic ring as described herein. In some of these embodiments, one or more of Rx, Rq and Rt is an ether group, such that the oxygen-containing heterocylic ring includes 2 oxygen atoms (forming, for example, 1,3-dioxolane, 1,3-dioxane or 1,4-dioxane, each being optionally unsubstituted).

In some of any of the embodiments described herein, A is the L-P, such that the phospholipid moiety is attached to the 5″ position of CBD. In some of these embodiments, B is hydrogen.

In some of any of the embodiments described herein. B is the L-P, such that the phospholipid moiety is attached to the 6 position of CBD. In some of these embodiments. A is hydrogen. In some of these embodiments, Rx is hydrogen. In other embodiments. Rx forms with part of the linking moiety a heteroalicyclic group as described herein.

Exemplary conjugates according to the present embodiments are presented in Table 1 in the Examples section that follows.

Exemplary processes of preparing conjugates as described herein are presented in the Examples section that follows.

Uses:

The compositions comprising particles encapsulating the CBD disclosed herein are used for treating medical conditions (e.g., diseases or disorders) that can benefit from treatment with and/or administration of CBD, which are also referred to herein as medical conditions (e.g., diseases or disorders) that are treatable by CBD.

The term “treating” or “treatment” refers to inhibiting, preventing or arresting the development of a pathology (disease, disorder or medical condition) and/or causing the reduction, remission, or regression of a pathology or a symptom of a pathology. Those of skill in the art will understand that various methodologies and assays can be used to assess the development of a pathology, and similarly, various methodologies and assays may be used to assess the reduction, remission or regression of a pathology.

The medical conditions according to the present embodiments are therefore those in which amelioration, reduction, remission or regression of a pathology or of symptoms thereof are effected by CBD.

In some embodiments, medical conditions treatable by, or which can benefit from treatment with, CBD, include medical conditions in which modulating (e.g., activating) an activity of a (e.g., central and/or peripheral) CB1 receptor is beneficial. As used herein, the term “subject” includes mammals, e.g., human beings at any age and of any gender. According to specific embodiments, the term “subject” refers to a subject who suffers from the pathology (disease, disorder or medical condition).

According to specific embodiments, the subject is a human.

Non-limiting examples of medical conditions that can benefit from treatment with CBD include epilepsy, anorexia, emesis, pain, inflammation, neurodegenerative disorders, nerve injury, glaucoma, osteoporosis, cognitive disorders, schizophrenia, Autism spectrum disorders (ASD), bipolar disorder, cardiovascular disorders, cancer, anxiety, stress, insomnia, glaucoma, inflammatory disease (e.g. inflammatory bowel disease, rheumatoid arthritis), infectious disease, high blood pressure, lung disease, autoimmune disease (e.g. fibromyalgia), obesity, and metabolic syndrome-related disorders.

According to specific embodiments, the medical condition is epilepsy, a neurodegenerative disease, a cognitive disease or disorder, a nerve injury, stroke, pain, inflammation or an infectious disease.

According to specific embodiments, the medical condition is a neurodegenerative disease, a nerve injury, stroke, inflammation, pain and an infectious disease.

According to specific embodiments, the disease is an inflammatory disease.

Inflammatory diseases include, but are not limited to, chronic inflammatory diseases and acute inflammatory diseases. Non-limiting examples of inflammatory diseases are provided infra.

Inflammatory Diseases Associated with Hypersensitivity

Examples of hypersensitivity include, but are not limited to, Type I hypersensitivity. Type II hypersensitivity, Type 111 hypersensitivity, Type IV hypersensitivity, immediate hypersensitivity, antibody mediated hypersensitivity, immune complex mediated hypersensitivity. T lymphocyte mediated hypersensitivity and DTH.

Type I or immediate hypersensitivity, such as asthma.

Type II hypersensitivity include, but are not limited to, rheumatoid diseases, rheumatoid autoimmune diseases, rheumatoid arthritis (Krenn V, et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J, et al., Immunol Res 1998; 17 (1-2):49), sclerosis, systemic sclerosis (Renaudineau Y, et al., Clin Diagn Lab Immunol. 1999 March; 6 (2):156); Chan O T, et al., Immunol Rev 1999 June; 169:107), glandular diseases, glandular autoimmune diseases, pancreatic autoimmune diseases, diabetes. Type I diabetes (Zimmet P. Diabetes Res Clin Pract 1996 October; 34 Suppl:S125), thyroid diseases, autoimmune thyroid diseases. Graves' disease (Orgiazzi J. Endocrinol Metab Clin North Am 2000 June; 29 (2):339), thyroiditis, spontaneous autoimmune thyroiditis (Braley-Mullen H. and Yu S. J Immunol 2000 Dec. 15; 165 (12):7262). Hashimoto's thyroiditis (Toyoda N, et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M, et al., J Reprod Immunol 1998 February; 37 (2):87), autoimmune anti-sperm infertility (Diekman A B, et al., Am J Reprod Immunol. 2000 March; 43 (3):134), repeated fetal loss (Tincani A, et al., Lupus 1998:7 Suppl 2:S107-9), neurodegenerative diseases, neurological diseases, neurological autoimmune diseases, multiple sclerosis (Cross A H, et al., J Neuroimmunol 2001 Jan. 1; 112 (1-2):1), Alzheimer's disease (Oron L, et al., J Neural Transm Suppl. 1997; 49:77), myasthenia gravis (Infante A J. And Kraig E, Int Rev Immunol 1999; 18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med Sci. 2000 April; 319 (4):234), myasthenic diseases, Lambert-Eaton myasthenic syndrome (Takamori M. Am J Med Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E, et al., Electroencephalogr Clin Neurophysiol Suppl 1999; 50:419); neuromyotonia, acquired neuromyotonia, arthrogryposis multiplex congenita (Vincent A, et al., Ann N Y Acad Sci. 1998 May 13; 841:482), cardiovascular diseases, cardiovascular autoimmune diseases, atherosclerosis (Matsuura E, el al., Lupus. 1998; 7 Suppl 2:S135), myocardial infarction (Vaarala O. Lupus. 1998; 7 Suppl 2:S132), thrombosis (Tincani A, et al., Lupus 1998; 7 Suppl 2:S107-9), granulomatosis, Wegener's granulomatosis, arteritis. Takayasu's arteritis and Kawasaki syndrome (Praprotnik S, et al., Wien Klin Wochenschr 2000 Aug. 25; 112 (15-16):660); anti-factor VIII autoimmune disease (Lacroix-Desmazes S, et al., Semin Thromb Hemost. 2000; 26 (2):157); vasculitises, necrotizing small vessel vasculitis, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3):178); antiphospholipid syndrome (Flamholz R, et al., J Clin Apheresis 1999:14 (4):171); heart failure, agonist-like β-adrenoceptor antibodies in heart failure (Wallukat G, et al., Am J Cardiol. 1999 Jun. 17; 83 (12A):75H), thrombocytopenic purpura (Moccia F. Ann Ital Med Int. 1999 April-June; 14 (2):114); hemolytic anemia, autoimmune hemolytic anemia (Efremov D G, et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A, el al., Gastroenterol Hepatol. 2000 January; 23 (1):16), celiac disease (Landau Y E, and Shoenfeld Y. Harefuah 2000 Jan. 16; 138 (2):122), autoimmune diseases of the musculature, myositis, autoimmune myositis. Sjogren's syndrome (Feist E, et al., Int Arch Allergy Immunol 2000 September; 123 (1):92); smooth muscle autoimmune disease (Zauli D, et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P, et al., Eur J Gastroenterol Hepatol. 1999 June; 11 (6):595).

Type IV or T cell mediated hypersensitivity, include, but are not limited to, rheumatoid diseases, rheumatoid arthritis (Tisch R. McDevitt H O. Proc Natl Acad Sci USA 1994 Jan. 18; 91 (2):437), systemic diseases, systemic autoimmune diseases, systemic lupus erythematosus (Datta S K., Lupus 1998; 7 (9):591), glandular diseases, glandular autoimmune diseases, pancreatic diseases, pancreatic autoimmune diseases. Type 1 diabetes (Castano L. and Eisenbarth G S. Ann. Rev. Immunol. 8:647); thyroid diseases, autoimmune thyroid diseases, Graves' disease (Sakata S, et al., Mol Cell Endocrinol 1993 March; 92 (1):77); ovarian diseases (Garza K M, et al., J Reprod Immunol 1998 February; 37 (2):87), prostatitis, autoimmune prostatitis (Alexander R B, et al., Urology 1997 December; 50 (6):893), polyglandular syndrome, autoimmune polyglandular syndrome, Type I autoimmune polyglandular syndrome (Hara T, et al., Blood. 1991 Mar. 1; 77 (5):1127), neurological diseases, autoimmune neurological diseases, multiple sclerosis, neuritis, optic neuritis (Soderstrom M, et al., J Neurol Neurosurg Psychiatry 1994 May; 57 (5):544), myasthenia gravis (Oshima M, et al., Eur J Immunol 1990 December; 20 (12):2563), stiff-man syndrome (Hiemstra H S, et al., Proc Natl Acad Sci USA 2001 Mar. 27; 98 (7):3988), cardiovascular diseases, cardiac autoimmunity in Chagas' disease (Cunha-Neto E, et al., J Clin Invest 1996 Oct. 15; 98 (8):1709), autoimmune thrombocytopenic purpura (Semple J W, et al., Blood 1996 May 15; 87 (10):4245), anti-helper T lymphocyte autoimmunity (Caporossi A P, el al., Viral Immunol 1998; 11 (1):9), hemolytic anemia (Sallah S, et al., Ann Hematol 1997 March; 74 (3):139), hepatic diseases, hepatic autoimmune diseases, hepatitis, chronic active hepatitis (Franco A, et al., Clin Immunol Immunopathol 1990 March; 54 (3):382), biliary cirrhosis, primary biliary cirrhosis (Jones D E. Clin Sci (Colch) 1996 November; 91 (5):551), nephric diseases, nephric autoimmune diseases, nephritis, interstitial nephritis (Kelly C J. J Am Soc Nephrol 1990 August; 1 (2):140), connective tissue diseases, ear diseases, autoimmune connective tissue diseases, autoimmune ear disease (Yoo T J, et al., Cell Immunol 1994 August; 157 (1):249), disease of the inner ear (Gloddek B, et al., Ann N Y Acad Sci 1997 Dec. 29; 830:266), skin diseases, cutaneous diseases, dermal diseases, bullous skin diseases, Pemphigus vulgaris, bullous pemphigoid and Pemphigus foliaceus.

Examples of delayed type hypersensitivity include, but are not limited to, contact dermatitis and drug eruption.

Examples of types of T lymphocyte mediating hypersensitivity include, but are not limited to, helper T lymphocytes and cytotoxic T lymphocytes.

Examples of helper T lymphocyte-mediated hypersensitivity include, but are not limited to Tₕ1 lymphocyte mediated hypersensitivity and Tₕ2 lymphocyte mediated hypersensitivity.

Autoimmune Diseases

Include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E, et al., Lupus. 1998; 7 Suppl 2:5135), myocardial infarction (Vaarala O. Lupus. 1998; 7 Suppl 2:S132), thrombosis (Tincani A, et al., Lupus 1998; 7 Suppl 2:5107-9). Wegener's granulomatosis, Takayasu's arteritis, Kawasaki syndrome (Praprotnik S, et al., Wien Klin Wochenschr 2000 Aug. 25:112 (15-16):660), anti-factor VIII autoimmune disease (Lacroix-Desmazes S, et al., Semin Thromb Hemost. 2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R, et al., J Clin Apheresis 1999; 14 (4):171), antibody-induced heart failure (Wallukat G, et al., Am J Cardiol. 1999 Jun. 17; 83 (12A):75H), thrombocytopenic purpura (Moccia F. Ann Ital Med Int. 1999 April-June; 14 (2):114; Semple J W, et al., Blood 1996 May 15; 87 (10):4245), autoimmune hemolytic anemia (Efremov D G, et al., Leuk Lymphoma 1998 January; 28 (3-4):285; Sallah S, et al., Ann Hematol 1997 March; 74 (3):139), cardiac autoimmunity in Chagas' disease (Cunha-Neto E, et al., J Clin Invest 1996 Oct. 15; 98 (8):1709) and anti-helper T lymphocyte autoimmunity (Caporossi A P, et al., Viral Immunol 1998; 11 (1):9).

Examples of autoimmune rheumatoid diseases include, but are not limited to rheumatoid arthritis (Krenn V, et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease. Type I diabetes, thyroid disease. Graves' disease, thyroiditis, spontaneous autoimmune thyroiditis. Hashimoto's thyroiditis, idiopathic myxedema, ovarian autoimmunity, autoimmune anti-sperm infertility, autoimmune prostatitis and Type I autoimmune polyglandular syndrome, diseases include, but are not limited to autoimmune diseases of the pancreas. Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases. Graves' disease (Orgiazzi J. Endocrinol Metab Clin North Am 2000 June; 29 (2):339; Sakata S, et al., Mol Cell Endocrinol 1993 March; 92 (1):77), spontaneous autoimmune thyroiditis (Braley-Mullen H. and Yu S. J Immunol 2000 Dec. 15:165 (12):7262), Hashimoto's thyroiditis (Toyoda N, et al., Nippon Rinsho 1999 August; 57 (8):1810), idiopathic myxedema (Mitsuma T. Nippon Rinsho. 1999 August; 57 (8):1759), ovarian autoimmunity (Garza K M, et al., J Reprod Immunol 1998 February; 37 (2):87), autoimmune anti-sperm infertility (Dickman A B, et al., Am J Reprod Immunol. 2000 March; 43 (3):134), autoimmune prostatitis (Alexander R B, et al., Urology 1997 December; 50 (6):893) and Type I autoimmune polyglandular syndrome (Hara T, et al., Blood. 1991 Mar. 1; 77 (5):1127).

Examples of autoimmune gastrointestinal diseases include, but are not limited to, chronic inflammatory intestinal diseases (Garcia Herola A, et al., Gastroenterol Hepatol. 2000 January; 23 (1):16), celiac disease (Landau Y E, and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, Pemphigus vulgaris, bullous pemphigoid and Pemphigus foliaceus.

Examples of autoimmune hepatic diseases include, but are not limited to, hepatitis, autoimmune chronic active hepatitis (Franco A, et al., Clin Immunol Immunopathol 1990 March; 54 (3):382), primary biliary cirrhosis (Jones D E. Clin Sci (Colch) 1996 November; 91 (5):551; Strassburg C P, et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross A H, et al., J Neuroimmunol 2001 Jan. 1; 112 (1-2):1), Alzheimer's disease (Oron L, et al., J Neural Transm Suppl. 1997; 49:77), myasthenia gravis (Infante A J. And Kraig E, Int Rev Immunol 1999; 18 (1-2):83; Oshima M, et al., Eur J Immunol 1990 December; 20 (12):2563), neuropathies, motor neuropathies (Kornberg A J. J Clin Neurosci. 2000 May; 7 (3):191); Guillain-Barre syndrome and autoimmune neuropathies (Kusunoki S. Am J Med Sci. 2000 April; 319 (4):234), myasthenia. Lambert-Eaton myasthenic syndrome (Takamori M. Am J Med Sci. 2000 April, 319 (4):204); paraneoplastic neurological diseases, cerebellar atrophy, paraneoplastic cerebellar atrophy and stiff-man syndrome (Hiemstra H S, et al., Proc Natl Acad Sci units S A 2001 Mar. 27; 98 (7):3988); non-paraneoplastic stiff man syndrome, progressive cerebellar atrophies, encephalitis. Rasmussen's encephalitis, amyotrophic lateral sclerosis. Sydeham chorea, Gilles de la Tourette syndrome and autoimmune polyendocrinopathies (Antoine J C. and Honnorat J. Rev Neurol (Paris) 2000 January; 156 (1):23): dysimmune neuropathies (Nobile-Orazio E, et al., Electroencephalogr Clin Neurophysiol Suppl 1999; 50:419); acquired neuromyotonia, arthrogryposis multiplex congenita (Vincent A, et al., Ann N Y Acad Sci. 1998 May 13; 841:482), neuritis, optic neuritis (Soderstrom M, et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E, et al., Int Arch Allergy Immunol 2000 September; 123 (1):92) and smooth muscle autoimmune disease (Zauli D, et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A, et al., Lupus 1998; 7 Suppl 2:5107-9).

Examples of autoimmune connective tissue diseases include, but are not limited to, ear diseases, autoimmune ear diseases (Yoo T J, et al., Cell Immunol 1994 August; 157 (1):249) and autoimmune diseases of the inner ear (Gloddek B, et al., Ann N Y Acad Sci 1997 Dec. 29; 830:266).

Examples of autoimmune systemic diseases include, but are not limited to, systemic lupus erythematosus (Erikson J, et al., Immunol Res 1998; 17 (1-2):49) and systemic sclerosis (Renaudineau Y, et al., Clin Diagn Lab Immunol. 1999 March; 6 (2):156); Chan O T, et al., Immunol Rev 1999 June; 169:107).

Allergic Diseases

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

Graft Rejection Diseases

Examples of diseases associated with transplantation of a graft include, but are not limited to, graft rejection, chronic graft rejection, subacute graft rejection, hyperacute graft rejection, acute graft rejection and graft versus host disease.

Infectious Diseases

As further described in details hereinbelow.

Cancer

As further described in details hereinbelow.

According to specific embodiments, the medical condition is a neurological disease.

As used herein, the phrase “neurological disease” refers to a disease of the brain, spine and/or the nerves that connect them.

Examples of neurological diseases or disorders, include, but are not limited to, epilepsy, convulsions, and seizure disorders, status epilepticus, a chemically-induced convulsion and/or seizure disorder, a febrile convulsion condition, a metabolic disturbance, a sustenance withdrawal condition, spasticity, skeletal muscle spasms, restless leg syndrome, anxiety, stress, multiple sclerosis, stroke, head trauma, spinal cord injury, (ALS), Parkinson's Disease, Huntington's Disease. Alzheimer's Disease, amyotrophic lateral sclerosis, neuropathic pain, myoclonus, schizophrenia, migraine, headaches and bipolar disorders.

According to specific embodiments, the disease is caused by a nerve injury, e.g. traumatic brain injury, spinal cord injury, and/or peripheral nerve injury.

According to one embodiment, the disease is a memory disease.

According to specific embodiments, the disease is a neurodevelopmental disorder such as autism or schizophrenia.

According to another embodiment, the disease is a behavioral disease such as schizophrenia, depression, anxiety, post-traumatic stress disorder (PTSD), attention deficit hyperactivity disorder, autism, Tourette's syndrome, obsessive compulsive disorder, as well as the neurobehavioral associated symptoms of degeneratives of the nervous system such as Parkinson's disease, essential tremor, Huntington's disease. Alzheimer's disease, multiple sclerosis and organic psychosis.

According to specific embodiments, the disease is a neurodegenarative disease such as, but not limited to, Alzheimer's disease. Parkinson's disease, multiple sclerosis. Huntington's disease, Tourette's syndrome, Alexander disease, Alper's disease. Amyotrophic lateral sclerosis, Ataxia telangiectasia, Batten disease (also known as Spielmeyer-Vogt-Sjogren-Batten disease). Bovine spongiform encephalopathy (BSE). Canavan disease, Cockayne syndrome, Corticobasal degeneration. Creutzfeldt-Jakob disease, HIV-associated dementia. Kennedy's disease. Krabbe disease, Lewy body dementia. Machado-Joseph disease (Spinocerebellar ataxia type 3). Multiple System Atrophy (MSA). Pelizaeus-Merzbacher Disease. Pick's disease. Primary lateral sclerosis, Refsum's disease, Sandhoff disease, Schilder's disease, Schizophrenia, Spielmeyer-Vogt-Sjogren-Batten disease (also known as Batten disease). Spinocerebellar ataxia (multiple types with varying characteristics), Spinal muscular atrophy, and Steele-Richardson-Olszewski disease.

According to specific embodiments, the disease is Alzheimer's disease.

According to specific embodiments, the disease is Parkinson's disease.

According to specific embodiments, the disease is epilepsy.

According to other specific embodiments, the disease is not epilepsy.

According to specific embodiments, the disease is a lung disease. Non-limiting examples of lung disease include virus-induced pneumonia, Chronic Obstructive Pulmonary Disorder (COPD), acute lung injury, pulmonary fibrosis, lung inflammation, bronchopulmonary dysplasia (BPD).

According to specific embodiments, the disease is an infectious disease.

As used herein, the term “infection” or “infectious disease” refers to a disease induced by a pathogen. Non-limiting specific examples of pathogens include, viral pathogens, bacterial pathogens e.g., intracellular mycobacterial pathogens (such as, for example, Mycobacterium tuberculosis), intracellular bacterial pathogens (such as, for example, Listeria monocytogenes), intracellular protozoan pathogens (such as, for example, Leishmania and Trypanosoma), parasitic diseases, fungal diseases, prion diseases.

Methods of analyzing infection are well known in the art and are either based on serology, protein markers, or nucleic acid assays.

According to some embodiments, infection is based on detection of unique sequences of virus RNA by NAAT such as real-time reverse-transcription polymerase chain reaction (rRT-PCR) with confirmation by nucleic acid sequencing when necessary.

According to specific embodiments, the disease is a viral infections disease. Non-limiting types of viral pathogens causing infectious diseases treatable according to specific embodiments of the present invention include, but are not limited to, retroviruses, circoviruses, parvoviruses, papovaviruses, adenoviruses, herpesviruses, iridoviruses, poxviruses, hepadnaviruses, picornaviruses, caliciviruses, togaviruses, flaviviruses, reoviruses, orthomyxoviruses, paramyxoviruses, rhabdoviruses, bunyaviruses, coronaviruses, arenaviruses, and filoviruses.

Non-limiting examples of viral infections include human immunodeficiency virus (HIV)-induced acquired immunodeficiency syndrome (AIDS), coronavirus, influenza, rhinoviral infection, viral meningitis, Epstein-Barr virus (EBV) infection, hepatitis A, B or C virus infection, measles, papilloma virus infection/warts, cytomegalovirus (CMV) infection, Herpes simplex virus infection, yellow fever, Ebola virus infection, rabies, etc.

According to specific embodiments, the disease is a virus-induced pneumonia. Non-limiting examples of viruses inducing pneumonia include influenza and corona viruses.

According to specific embodiments, the disease is a Coronavirus infection.

According to specific embodiments, a clinical manifestation of Coronavirus infection includes symptoms selected from the group consisting of inflammation in the lung, alveolar damage, fever, cough, shortness of breath, diarrhea, organ failure, pneumonia and/or septic shock.

As used herein, “Coronavirus” refers to enveloped positive-stranded RNA viruses that belong to the family Coronaviridae and the order Nidovirales.

Examples of Corona viruses which are contemplated herein include, but are not limited to 229E, NL63, OC43, and HKU1 with the first two classified as antigenic group 1 and the latter two belonging to group 2, typically leading to an upper respiratory tract infection manifested by common cold symptoms.

However. Coronaviruses, which are zoonotic in origin, can evolve into a strain that can infect human beings leading to fatal illness. Thus particular examples of Coronaviruses contemplated herein are SARS-CoV, Middle East respiratory syndrome Coronavirus (MERS-CoV), and the recently identified SAR-CoV-2 [causing 2019-nCoV (also referred to as “COVID-19”)].

It would be appreciated that any Coronavirus strain is contemplated herein even though SAR-CoV-2 is emphasized in a detailed manner.

According to specific embodiments, the disease is a SAR-CoV-2 infection.

According to specific embodiments, the disease is cancer.

Cancers which may be treated by some embodiments of the invention can be any solid or non-solid tumor, cancer metastasis and/or a pre-cancer.

According to specific embodiments, the cancer is a malignant cancer.

Examples of cancer include but are not limited to, carcinoma, blastoma, sarcoma and lymphoma. More particular examples of such cancers include, but are not limited to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell. Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukenia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphohlastic, acute lymphohlastic pre-B cell, acute lymphoblastic T cell leukemia, acute-megakaryoblastic, monocytic, acute myelogenous, acute myeloid, acute myeloid with eosinophilia. B cell, basophilic, chronic myeloid, chronic, B cell, eosinophilic. Friend, granulocytic or myelocytic, hairy cell, lymphocytic, megakaryohlastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, ostcomycloma, ostcosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell. Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

According to specific embodiments, the cancer is a pre-malignant cancer.

Pre-cancers are well characterized and known in the art (refer, for example, to Berman J J. and Henson D E., 2003. Classifying the pre-cancers: a metadata approach. BMC Med Inform Decis Mak. 3:8). Examples of pre-cancers include, but are not limited to, acquired small pre-cancers, acquired large lesions with nuclear atypia, precursor lesions occurring with inherited hyperplastic syndromes that progress to cancer, and acquired diffuse hyperplasias and diffuse metaplasias. Non-limiting examples of small pre-cancers include HGSIL (High grade squamous intraepithelial lesion of uterine cervix), AIN (anal intraepithelial neoplasia), dysplasia of vocal cord, aberrant crypts (of colon), PIN (prostatic intraepithelial neoplasia).

Non-limiting examples of acquired large lesions with nuclear atypia include tubular adenoma, AILD (angioimmunoblastic lymphadenopathy with dysproteinemia), atypical meningioma, gastric polyp, large plaque parapsoriasis, myelodysplasia, papillary transitional cell carcinoma in-situ, refractory anemia with excess blasts, and Schneiderian papilloma. Non-limiting examples of precursor lesions occurring with inherited hyperplastic syndromes that progress to cancer include atypical mole syndrome, C cell adenomatosis and MEA. Non-limiting examples of acquired diffuse hyperplasias and diffuse metaplasias include Paget's disease of bone and ulcerative colitis.

According to some of any of the embodiments described herein, the medical condition is or comprises pain, including neuropathic pain and neurogenic pain.

As used herein, the term “pain” encompasses both acute and chronic pain. As used herein, the term “acute pain” means immediate, generally high threshold, pain brought about by injury such as a cut, crush, burn, or by chemical stimulation such as that experienced upon exposure to capsaicin, the active ingredient in chili peppers. The term “chronic pain,” as used herein, means pain other than acute pain and includes, without limitation, neuropathic pain, visceral pain, fibromyalgia pain, inflammatory pain, headache pain, muscle pain and referred pain.

The cells from which the particles were obtained according to specific embodiments of the present invention may be autologous or non-autologous to the subject; they can be syngeneic or non-syngeneic: allogeneic or xenogeneic to the subject; each possibility represents a separate embodiment of the present invention.

According to specific embodiments, the cells from which the particles were obtained are autologous to the subject.

According to specific embodiments, the cells from which the particles were obtained are non-autologous to the subject.

According to some of any of the embodiments described herein, the cell-derived particle features a biological activity, as described herein in any of the respective embodiments.

According to some of any of the embodiments described herein, the cell-derived particle and the CBD act in synergy.

By “act in synergy” it is meant that the therapeutic activity exhibited by the particle that encapsulate CBD, as described herein in any of the respective embodiments, is higher than the additive activity of the particle and the CBD when used alone. The therapeutic activity can any of the activities described herein in the context of the uses of the compositions of the present embodiments. For example, the therapeutic activity can be an anti-inflammatory activity, and anti-viral activity, or a treatment of any of the medical conditions described herein. Determining the therapeutic activity can be performed by any method known in the art, some of which are exemplified in the Examples section that follows.

Synergy can be determined by methods known in the art. In some embodiments, synergy is determined by means of an isobologram, as widely described in the art.

In some embodiments, the synergistic effect provided by a composition as described herein allows using sub-therapeutic doses of each component, for example, sub-therapeutic dose of CBD.

Pharmaceutical Compositions:

In any of the methods and uses described herein, the composition comprising the particles encapsulating the CBD can be administered either per se or, as a part of a pharmaceutical composition that further comprises a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the particles encapsulating CBD described herein accountable for the biological effect.

Hereinafter, the term “pharmaceutically acceptable carrier” refers to a carrier or a diluent that does not cause significant irritation to a subject and does not abrogate the biological activity and properties of the administered compound. Examples, without limitations, of carriers are propylene glycol; saline; emulsions; buffers; culture medium such as DMEM or RPMI; hypothermic storage medium containing components that scavenge free radicals, provide pH buffering, oncotic/osmotic support, energy substrates and ionic concentrations that balance the intracellular state at low temperatures; and mixtures of organic solvents with water.

Typically, the pharmaceutical carrier preserves the number of particles (e.g. is not reduced by more than 90%) in the composition for at least 24 hours, at least 48 hours or even at least 96 hours.

Herein the term “excipient” refers to an inert substance added to a pharmaceutical composition to further facilitate administration of an active agent and/or maintain stability or integrity at a pre-determined temperature for a suitable period of time before administration. Examples, without limitation, of excipients include albumin, plasma, serum and cerebrospinal fluid (CSF), antioxidants such as N-Acetylcysteine (NAC) or resveratrol.

According to a preferred embodiment of the present invention, the pharmaceutical carrier is an aqueous solution of buffer or a culture medium such as DMEM.

Techniques for formulation and administration of drugs may be found in “Remington's Pharmaceutical Sciences.” Mack Publishing Co., Easton, Pa, latest edition, which is incorporated herein by reference.

The composition comprising the CBD encapsulated in the particle disclosed herein can be administered to the treated individual using a variety of routes, the nature of which depends on the target cells or tissue. For example, the composition can be administered intranasally (e.g. by inhalation), intrathecally (into the spinal canal, or into the subarachnoid space), arterially, intradermally (by absorption e.g. through the skin), intramuscularly, intraperitoneally, intravenously, subcutaneously, ocularly, sublingually, orally (by ingestion), intracerelrally. Other modes of administration are also contemplated.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

For injection, the composition may be formulated in aqueous solutions, preferably in physiologically compatible buffers such as Hank's solution, Ringer's solution, or physiological salt buffer and additional agents as further described herein.

For transmucosal administration, penetrants appropriate to the barrier to be permeated are used in the formulation. Such penetrants are generally known in the art.

According to specific embodiments, the composition is administered non-invasively e.g. orally, intranasally.

For administration by inhalation, the composition is conveniently delivered in the form of an aerosol spray presentation from a pressurized pack or a nebulizer with the use of a suitable propellant. e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

For oral administration, the composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptably polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (particles encapsulating CBD) effective to prevent, alleviate or ameliorate symptoms of a disorder or prolong the survival of the subject being treated.

Determination of a therapeutically effective amount is well within the capability of those skilled in the art.

For any preparation used in the methods of the invention, the therapeutically effective amount or dose can be estimated initially from in-vitro and cell culture assays. Preferably, a dose is formulated in an animal model to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. Further information may be obtained from clinical studies—see for example Salem H K et al., Stem Cells 2010; 28:585-96; and Uccelli et al. Lancet Neurol. 2011; 10:649-56). The dosage may vary depending upon the dosage form employed and the route of administration utilized. The exact formulation, route of administration and dosage can be chosen by the individual physician in view of the patient's condition, (see e.g., Fingl, et al., 1975, in “The Pharmacological Basis of Therapeutics”. Ch. 1 p. 1).

Dosage amount and interval may be adjusted individually to provide levels of the active ingredients which are sufficient to effectively treat the disease. Dosages necessary to achieve the desired effect will depend on individual characteristics and route of administration.

An exemplary dose of particles (e.g. exosomes) that may be administered (e.g. intranasally) per treatment may be between 1×10⁶-1×10²⁰ and more preferably between 1×10⁹-1×10¹⁵ for a 70 kg human.

An exemplary dose of CBD that may be administered may be between 1-50 mg/kg/day.

Depending on the severity and responsiveness of the condition to be treated, dosing can be of a single or a plurality of administrations, with course of treatment lasting from several days to several weeks or months depending when diminution of the disease state is achieved.

The amount of the active ingredients (particles encapsulating CBD) to be administered will, of course, be dependent on the individual being treated, the severity of the affliction, the manner of administration, the judgment of the prescribing physician, etc. The dosage and timing of administration will be responsive to a careful and continuous monitoring of the individual changing condition.

Following administration, the particles may be tracked in order to ensure they have reached the target site. This may be carried out using gold nanoparticle, see for example International Patent Application Publication No. WO2013186735.

The composition comprising particles encapsulating CBD of the present invention, in at least some embodiments, may be prepackaged in unit dosage forms in a syringe ready for use. The syringe may be labeled with the name of the composition e.g. particles and their source. The labeling may also comprise information related to the function of the composition. The syringe may be packaged in a packaging which is also labeled with information regarding the composition.

The composition of some embodiments of the invention can be administered to the subject as a single treatment or in combination with other established (e.g. gold standard) or experimental therapeutic regimen to treat the disease including, but not limited to analgesics, chemotherapeutic agents, radiotherapeutic agents, cytotoxic therapies (conditioning), hormonal therapy, antibodies, antibiotics, antimetabolites small molecule agents and precursors of neurotransmitter molecules such as L-DOPA, anti-inflammatory drugs, immune-suppressive drugs, neurotrasnmitters, neurohormones, toxins, and other treatment regimens (e.g., surgery) which are well known in the art. Additionally, or alternatively, the composition, in at least some embodiments, may be co-administered with other cells capable of alleviating at least one symptom of the disease.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

In any of the methods and uses as described herein, the composition or a pharmaceutical composition comprising same can be used in combination with an additional agent that is usable in the treatment of the medical condition.

Embodiments of the present invention further relate to kits comprising the particles and the CBD, as described herein in any of the respective embodiments, optionally packaged separately within the kit. In some embodiments, the kit can further comprise instructions to prepare a composition as described herein, for example, by methods as described herein (although other methods are also contemplated). In some embodiments, the kit is identified for use in treating a medical condition as described herein in any of the respective embodiments.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5.000 nucleotides, alternatively, less than 1 in 10,000 nucleotides.

As used herein, the term “alkyl” describes an aliphatic hydrocarbon including straight chain and branched chain groups. Preferably, the alkyl group has 1 to 20 carbon atoms, and more preferably 1 to 10 carbon atoms. Whenever a numerical range; e.g., “1 to 10”, is stated herein, it implies that the group, in this case the alkyl group, may contain 1 carbon atom, 2 carbon atoms, 3 carbon atoms, etc., up to and including 10 carbon atoms. In the context of the present invention, a “long alkyl” is an alkyl having at least 10 carbon atoms in its main chain (the longest path of continuous covalently attached atoms). In the context of the present invention, a “medium alkyl” is an alkyl having from 5 to 9 carbon atoms in its main chain (the longest path of continuous covalently attached atoms). A short alkyl therefore has 4 or less main-chain carbons. The alkyl can be substituted or unsubstituted. When substituted, the substituent can be, for example, an alkyl, an alkenyl, an alkynyl, a cycloalkyl, an aryl, a heteroaryl, a halide, an amine, a hydroxyl, a thiol, an alkoxy and a thioalkoxy, as these terms are defined herein.

The alkyl group can be an end group, as this phrase is defined herein, wherein it is attached to a single adjacent atom, or a linking group, as this phrase is defined herein, which connects two or more moieties via at least two carbons in its chain. When the alkyl is a linking group, it is also referred to herein as “alkylene” or “alkylene chain”.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or marc substituents, as described hereinabove.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. Substituted heteroalicyclic may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, hydroxy, alkoxy and thioalkoxy. Representative examples are piperidine, piperazine, tetrahydrofurane, tetrahydropyrane, morpholino and the like.

Piperidine and piperazine are exemplary nitrogen-containing heteroalicylic.

The term “hydroxy”, as used herein, refers to an —OH group.

The term “alkoxy” refers to a —OR′ group, were R′ is alkyl, aryl, heteroalicyclic or heteroaryl.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted by one or more substituents, as described hereinabove.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted by one or more substituents, as described hereinabove. Representative examples of nitrogen-containing heterocyclics include imidazole, thiadiazole, pyridine, pyrrole, oxazole, indole, purine and the like.

As used herein, the terms “halo” and “halide”, which are referred to herein interchangeably, describe an atom of a halogen, that is fluorine, chlorine, bromine or iodine, also referred to herein as fluoride, chloride, bromide and iodide.

The term “haloalkyl” describes an alkyl group as defined above, further substituted by one or more halide(s).

The term “alkylene” as used herein describes a —(CR′R″)f-, wherein R′ and R″ are as described herein, and f is an integer from 1 to 20, or from 1 to 10.

The term “thiol” describes a —SH group.

The term “thioalkoxy” describes both an —S-alkyl group, and an —S-cycloalkyl group, as defined herein.

The term “cyano” describes a —C≡N group.

The term “carbonyl” describes a —C(═O)—R′ group, where R′ is hydrogen, alkyl, cycloalkyl, aryl, heteroaryl (bonded through a ring carbon) or heteroalicyclic (bonded through a ring carbon) as defined herein.

The term “thiocarbonyl” describes a —C(═S)—R′ group, where R′ is as defined herein.

The term “O-carbamyl” describes an —OC(═O)—NR′ R″ group, where R′ is as defined herein and R″ is hydrogen, alkyl, cycloalkyl, aryl, heteroaryl (bonded through a ring carbon) or heteroalicyclic (bonded through a ring carbon) as defined herein.

The term “N-carbamyl” describes an R′ OC(═O)—NR″— group, where R′ and R″ are as defined herein.

The term “O-thiocarbamyl” describes an —OC(═S)—NR′ R″ group, where R′ and R″ are as defined herein.

The term “N-thiocarbamyl” describes an R′OC(═S)NR′— group, where R′ and R″ are as defined herein.

The term “amide” describes a —C(═O)—NR′ R″ group, where R′ and R″ are as defined herein.

The term “carboxy” describes a —C(═O)—O—R′ groups, where R′ is as defined herein. When R′ is H, this term is also referred to herein as carboxylic acid. When R′ is alkyl, cycloalkyl or aryl, this term is also referred to herein as carboxylate.

The term “sulfonyl” group describes an —S(═O)₂—R′ group, where R′ is as defined herein.

The term “halogen” or “halo” describes fluoro, chloro, bromo or iodo atom.

As used herein, the term “amine” describes both a —NR′ R″ group and a —NR′— group, wherein R′ and R″ are each independently hydrogen, alkyl, cycloalkyl, aryl, as these terms are defined hereinbelow.

The amine group can therefore be a primary amine, where both R′ and R″ are hydrogen, a secondary amine, where R′ is hydrogen and R″ is alkyl, cycloalkyl or aryl, or a tertiary amine, where each of R′ and R″ is independently alkyl, cycloalkyl or aryl.

Alternatively, R′ and R″ can each independently be hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, carbonyl. C-carboxylate. O-carboxylate, N-thiocarbamate, O-thiocarbamate, urea, thiourea, N-carbamate, O-carbamate, C-amide, N-amide, guanyl, guanidine and hydrazine.

The term “amine” is used herein to describe a —NR′ R″ group in cases where the amine is an end group, as defined hereinunder, and is used herein to describe a —NR′— group in cases where the amine is a linking group.

Herein throughout, the phrase “end group” describes a group (a substituent) that is attached to another moiety in the compound via one atom thereof.

The phrase “linking group” describes a group (a substituent) that is attached to another moiety in the compound via two or more atoms thereof.

The term “ether” as used herein describes an R′—O—R″ group, wherein R′ and R″ are each independently an alkyl or alkylene, cycloalkyl or aryl.

According to some of any of the embodiments described herein, any of the conjugates prepared or provided according to the present embodiments can be in a form of a pharmaceutically acceptable salt thereof. In the context of these embodiments, the term “conjugate” is also referred to simply as a “compound”.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, and/or to improve its stability, while not abrogating the biological activity and properties of the administered compound. A pharmaceutically acceptable salt of a compound as described herein can alternatively be formed during the synthesis of the compound, e.g., in the course of isolating the compound from a reaction mixture or re-crystallizing the compound.

In the context of some of the present embodiments, a pharmaceutically acceptable salt of the compounds described herein may optionally be an acid addition salt comprising at least one basic group (e.g., an amine-containing group) of the compound which is in a positively charged form (e.g., wherein the basic group is protonated), in combination with at least one counter-ion, derived from the selected base, that forms a pharmaceutically acceptable salt.

The acid addition salts of the compounds described herein may therefore be complexes formed between one or more basic groups of the compound and one or more equivalents of an acid.

Depending on the stoichiometric proportions between the charged group(s) in the compound and the counter-ion in the salt, the acid additions salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt would be an ammonium cation and an acid addition salt thereof.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

In the context of some of the present embodiments, a pharmaceutically acceptable salt of the conjugates described herein may optionally be a salt comprising at least one phosphate group of the phospholipid which is in a negatively charged form (e.g., wherein the phosphate group is de-protonated), in combination with at least one anion, that forms a pharmaceutically acceptable salt.

The present embodiments further encompass any enantiomers, diastereomers, prodrugs, solvates, hydrates and/or pharmaceutically acceptable salts of the conjugates described herein.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an R- or an S-configuration and any combination, and compounds according to some embodiments of the present invention, can have any their chiral centers exhibit an R- or an S-configuration.

The term “diastereomers”, as used herein, refers to stereoisomers that are not enantiomers to one another. Diastereomerism occurs when two or more steroisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

The term “prodrug” refers to an agent, which is converted into the active compound (the active parent drug) in vivo. Prodrugs are typically useful for facilitating the administration of the parent drug. They may, for instance, be bioavailable by oral administration whereas the parent drug is not. A prodrug may also have improved solubility as compared with the parent drug in pharmaceutical compositions. Prodrugs are also often used to achieve a sustained release of the active compound in vivo.

The term “solvate” refers to a complex of variable stoichiometry (e.g., di-, tri-, tetra-, penta-, hexa-, and so on), which is formed by a solute (the compound of the present invention) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”. John Wiley and Sons. Baltimore. Md. (1989); Perbal, “A Practical Guide to Molecular Cloning”, John Wiley & Sons. New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press. New York (1998); methodologies as set forth in U.S. Pat. Nos. 4,666,828; 4,683,202; 4,801,531; 5,192,659 and 5,272,057; “Cell Biology: A Laboratory Handbook”, Volumes I-TIT Cellis, J. E., ed. (1994); “Culture of Animal Cells—A Manual of Basic Technique” by Freshney. Wiley-Liss. N. Y. (1994), Third Edition; “Current Protocols in Immunology” Volumes I-III Coligan J. E., ed. (1994); Stites et al. (eds). “Basic and Clinical Immunology” (8th Edition). Appleton & Lange. Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames. B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal. B., (1984) and “Methods in Enzymology” Vol. 1-317. Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### MSC-Derived Exosomes Encpaulating CBD as Treatment for Epilepsy

The pilocarpine-induced epilepsy is a well-established modal that causes chronic epilepsy in mice. Specifically, a single ant dose of pilocarpine (340 mg/kg, Sigma. Israel) is injected subcutaneously. Status epilepticus (SE) is defined as a sustained series of generalized tonic-clonic convulsions (stage V). Diazepam (4 mg/kg. Teva, Israel) is injected intraperitoneally 40 minutes following the onset of SE to terminate seizures. To minimize peripheral muscarinic stimulation, methyl□scopolamine (1 mg/kg, Sigma, Israel) is administered subcutaneously prior to pilocarpine injection. In this model, following a latent period of 1-2 weeks, the initial SE then triggers the process of epileptogenesis, leading to chronic epilepsy and spontaneous recurrent seizures (SRS). Only mice developing clinical SE after pilocarpine injection, including whole body tonic-clonic seizures with loss of posture or jumping are subsequently included in additional phenotypic and correlative analyses. Naive mice are used as control mice for the described experiments.

To evaluate the therapeutic effect of MSC-derived exosomes encapsulating CBD. MSC-derived exosomes are prepared as previously described [Perets N et al., (2019) Nano Lett. 19(6):3422-3431; Perets N et al., (2018) Mol Autism. 9:57] loaded with CBD and administered intranasally to epileptic mice.

Mice are divided into four groups as follows: Group A—Control (PBS) treated mice, Group B—treated with CBD (100 mg/kg for 10 days) alone. Group C—treated with MSC-derived exosomes (10⁹ particles/2 μL for 4 days) alone, Group D—treated with MSC-derived exosomes encapsulating CBD (10⁹ particles/2 μL for 4 days).

Following, electroencephalography (EEG, e.g. implantable telemetric EEG transmitters coupled with a video recording system as described in Chang P et al. [J Neurosci Methods. (2011) 201(1):106-15] is recorded; and pro-inflammatory cytokine production and microgliosis in the hippocampus are analyzed.

### Example 2

### MSC-Derived Exosomes Encpaulating CBD as Treatment for Alzheimer's Disease

The 5XFAD mouse model is a well-established model for Alzheimer's disease. Specifically, 5XFAD mice are one of the most early-onset and aggressive amyloid mouse models [Oakley H, et al. (2006) J Neurosci 26: 10129-10140]. These mice co-overexpress and coinherit neuron-specific transgenes with five familial AD (FAD) mutations, in human APP and PSI, acting together to additively increase levels of cerebral A peptides. Thus, 5XFAD mice start to develop detectable amyloid deposits as early as 2 months of age, first in the subiculum and in layer 5 of the neocortex with a rapid increase across age consistent with dramatically accelerated A42 generation [Oakley et al.].

To evaluate the therapeutic effect of MSC-derived exosomes encapsulating CBD, MSC-derived exosomes are prepared as previously described [Perets N et al., (2019) Nano Lett. 19(6):3422-3431; Perets N et al., (2018) Mol Autism. 9:57], loaded with CBD and administered intranasally to 5×FAD mice.

Mice are divided into four groups as follows: Group A—Control (PBS) treated mice, Group B—treated with CBD (100 mg/kg for 10 days) alone, Group C—treated with MSC-derived exosomes (10⁹ particles/2 μL for 4 days) alone. Group D—treated with MSC-derived exosomes encapsulating CBD (10⁹ particles/2 μL for 4 days).

Following, mice are monitored using e.g. the following tests.

Elevated Plus Maze—The elevated plus maze is generally used for the assessment of anxiety-related behavior. A plus-shaped maze containing two dark and enclosed arms and two open and lit arms, elevated 100 cm above ground, is used. The arms are 30×5 cm with a 5×5 cm center area, and the walls of the closed arms are 40 cm high. Mice are placed in the center of the maze, tracked for 5 minutes with a video camera, and then returned to their home cage. Time spent in the open arms is measured using Ethovision video tracking system.

Y-Maze—Forced alternation Y-maze is performed to assess spatial memory as previously described [Volkman. R., et al. (2019) Front. Neurosci. 13]. The test is conducted in a white, Perspex Y-shape apparatus with arm length of 38 cm, width of 5 cm, and height of 15 cm. The test comprises a sample trial and a test trial. In the sample trial, mice are placed at the end of one arm of the maze facing the wall, while one arm of the maze is blocked, and mice can explore the two arms of the maze for 5 minutes. The sample trial is followed by a 5 minutes inter-trial interval. In the test trial, the mice are returned to the maze with all arms open for additional 5 minutes. Novel arm exploration time is measured for the duration of test trial.

Morris Water Maze—Mice are assessed for memory retention and cognition in the Morris water maze (MWM) [Vorhees. C. V. & Williams, M. T. (2006) Nat. Protoc. 1: 848-858]. The test comprises a large pool of water with visual cues and a hidden platform located at the same quadrant throughout the learning phase (quadrant 1). Mice are released from a different quadrant in the pool four times per day for 60 seconds trials during the four day learning period. Latency to reach the platform is calculated each day as a mean of all trials. During the learning phase, mice that do not find the platform are encouraged towards the platform and left untouched for 30 seconds. Mice that fail to find the platform are scored as having reached the platform in 60 seconds. On the fifth day, the platform is removed and mice are released from the opposite side for a 60 seconds probe trial. Time spent in the platform quadrant 1 (Q1) is tracked using Ethovision 11.5 software.

Brain preparation for neuropathology analysis—Mice designated for histology analysis are injected with a mixture of Ketamine/Xylazine (100/10 mg/kg, respectively) IP. Following, using an electric pump mice are intracardially perfused with PBS followed by ice-cold 4% paraformaldehyde (PFA) in PBS. The brains are removed and post-fixed in 4% PFA at 4° C., for 24 hours and then cryopreserved in 30% sucrose. Subsequently, brains are stored in PBS with 0.02% sodium azide (Sigma-Aldrich) at 4° C. until immunohistochemical processing. Mice designated for brain dissection are sacrificed using CO₂. The brain is removed and quickly dissected on ice for left/right prefrontal cortex (PFC), left/right hippocampus and cerebellum. Tissues are snap frozen in liquid nitrogen and transferred to −80° C. until analysis. These samples are used for DNA, RNA and protein analysis.

Immunoblotting—Proteins are extracted from cells or brain tissue as follows: The cells are washed twice with PBS and re-suspended in a lysis buffer containing 250 mM sucrose, 25 mM Tris/HCl, pH 6.8, 1 mM EDTA, 0.05% digitonin, 1 mM dithiothreitol (DTT), 0.1 mM phenylmethylsulfonylfluoride, 1:100 v/v complete protease inhibitor cocktail (Roche). For brain samples, the same lysis buffer and protease inhibitor cocktail are used. Samples are macerated gently in their vials. Both cell and brain samples are incubated one hour on ice. Samples are then centrifuged at 14,000 RPM for 20 minutes at 4° C. Protein levels are determined using BCA kit (Thermofisher). Supernatants are stored at −80° C. until further use. Proteins are separated by 8-15% sodiumdodecyl sulfate (SDS) polyacrylamide gel electrophoresis and transferred to nitrocellulose membranes. The membranes are blocked using 5% Bovine serum albumin (BSA) dissolved in PBS-Tween (PBST). The membranes are probed overnight at 4° C. with rabbit anti-synaptophysin (1:1000, Abcam). Following PBST wash, membranes are incubated with secondary antibodies: goat anti-mouse or goat anti-rabbit IRDye®800CW/680CW (1:10.000. Licor) for 1 hour at room temperature. The membranes are then developed with Odyssey Imager (model 9120, Licor). As a control for protein loading, blots are subsequently probed for mouse anti β-actin (1:1,000, Sigma-Aldrich) using the same procedures. Data is calculated as the ratio of mean target protein intensity to β-actin intensity. Densitometric analysis of Western blots is performed using Odyssey 2.1 software (Licor) to measure the area and density of protein bands.

Immunohistochemistry staining—Perfused brains are dried and snap frozen in 2-Methylbutane (Sigma-Aldrich) in liquid Nitrogen. Brains are sectioned (10 μm) using a cryostat and mounted directly onto slides for analysis. For immunohistochemistry, slides are incubated with blocking solution (5% goat serum. 1% BSA. 0.05% Triton-X in PBS) for 1 hour at room temperature (RT), following by incubated overnight at 4° C. with the following primary antibodies: rabbit anti-GFAP (1:500, ab7260, Abcam), rabbit anti-IBAI (1:500, ab178847, Abcam). Following, sections are incubated with secondary antibodies: goat anti-rabbit Alexa 488 (1:700, Invitrogen) for 1 hour at RT. The nuclei are stained with DAPI (1:1000. Sigma-Aldrich). For microscopic analysis, Lcica SP5 confocal laser scanning microscope is used (Leica microsystems. Wetzlar. Germany). Intensity of fluorescence is measured using ImageJ software (ImageJ software). At least three brains for each group are used for quantification.

For Thioflavin S (ThioS, Sigma-Aldrich) staining, following the blocking step, slides are incubated for 8 minutes with 0.01% ThioS solution in 50% ethanol. Slides are then briefly incubated twice for 10 seconds with 80% ethanol, and washed twice with double distilled water (DDW).

Real-time PCR—Hippocampal RNA is extracted using RNeasy Mini Kit (Qiagen) as previously described [Rio, D. C., et al. (2010) Cold Spring Harb. Protoc. 2010, pdb.prot5439]. RNA is reverse transcribed to complementary DNA (cDNA) using verso cDNA synthesis kit (Thermo Fisher Scientific). Semi-quantitative PCR is performed on the Step-One Real time PCR (RT-PCR) system using Syber-Green Master mix (Thermo Fisher Scientific) and the custom designed primers. Threshold cycle values are determined in triplicates and presented as average compared with Actin. Fold changes are calculated using the ^{2ΔCT} method.

### Example 3

### MSC-Derived Exosomes Encpaulating CBD as Treatment for Corona Virus

The lungs are the organs most affected by SAR-CoV-2 [causing 2019-nCoV (also referred to as “COVID-19”)], because the virus accesses host cells via the enzyme ACE2, which is most abundant in the alveolar cells of the lungs. SAR-CoV-2 induced pneumonia may rapidly progress to acute respiratory distress syndrome causing respiratory failure, septic shock, or multi-organ failure.

The therapeutic effect of MSC-derived exosomes encapsulating CBD is first evaluated in-vitro using standard assays to measure the effects on the cytotoxicity, virus yield and infection rates of (see Wang M et al. (2020) Cell Res. 30(3):269-271). Specifically, the cytotoxicity of CBD. MSC-derived exosomes and MSC-derived exosomes encapsulating CBD in Vero E6 cells (ATCC-1586) is determined using the CCK8 assay. Then, Vero E6 cells are infected with SAR-CoV-2 at e.g. a multiplicity of infection (MOI) of 0.05 in the presence of varying concentrations of CBD. MSC-derived exosomes and MSC-derived exosomes encapsulating CBD. DMSO is used in the controls. Efficacies are evaluated by quantification of viral copy numbers in the cell supernatant via quantitative real-time RT-PCR (qRT-PCR) and confirmed with visualization of virus nucleoprotein (NP) expression through immunofluorescence microscopy at 48 hours post infection (p.i.) (cytopathic effect is not obvious at this time point of infection).

In the next step, the therapeutic effect of MSC-derived exosomes encapsulating CBD is evaluated in-vivo, using e.g. the mouse-adapted MA15 SARS-CoV which is a well-established model that causes a dose dependent lung disease and significant morbidity and mortality in BALB/C mice (see e.g. Kumaki Y, et al. (2011) Antiviral Res. 2011, 89(1):75-82).

Mice are divided into four groups as follows: Group A—Control (PBS) treated mice, Group B—treated with CBD (100 mg/kg for 10 days) alone, Group C—treated with MSC-derived exosomes (10⁹ particles/2 μL for 4 days) alone. Group D—treated with MSC-derived exosomes encapsulating CBD (10⁹ particles/2 μL for 4 days).

Mice are monitored daily for weight loss and survival.

Lung tissue histopathology in is examined on e.g. day 2 and 10.

### Example 4

### Design and Preparation of Exemplary Modified CBD

CBD Structural Docking Studies in CB1R:

The present inventors have used Auto-Dock vina [Vina, A. J. Comput. Chem 31.2 (2010): 455-461] to perform docking analyses on CBD inside CB1 receptor [structure adopted from www(dot)resb(dot)org/structure/5TGZ], in order to find an available atom through which a moiety could be bound to CBD in a manner that would not affect its binding and activity inside the receptor.

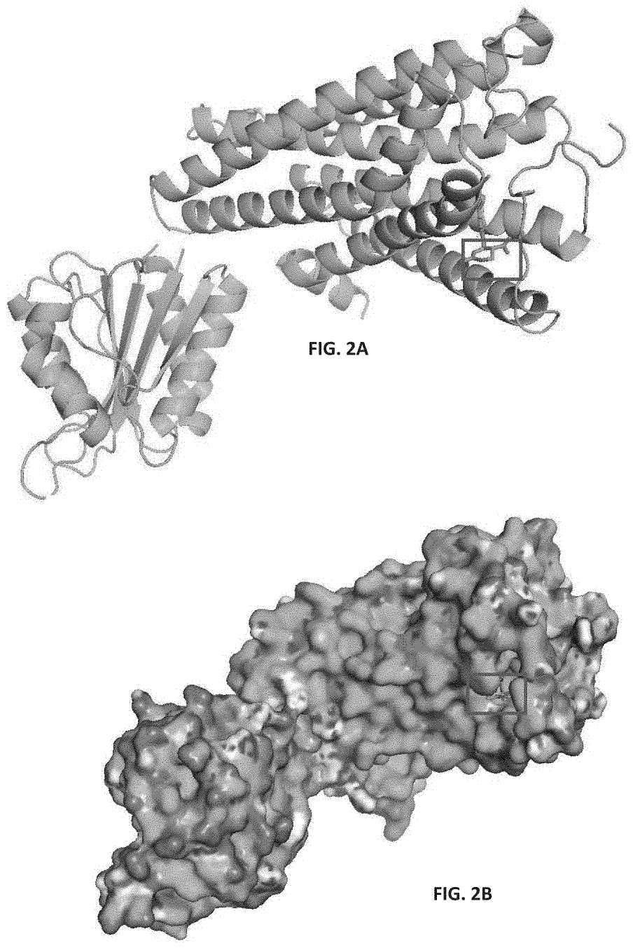

The data obtained in this analysis are shown in FIG. 1A (showing the tested positions in CBD) and FIG. 1B (showing the obtained values), and the cartoon and surface structures on CB1 receptor (cyan) with CBD (green) inside the active site as predicted using the AutoDock vina are shown in FIGS. 2A-B, respectively.

As can be seen in FIGS. 2A-B, the highest potential position output (table 1: mode 1) demonstrated that carbon ‘6’ (see. FIG. 1A) in the CBD structure is positioned outside the receptor surface when CBD interacts with the receptor thus exhibiting minimal interactions with the receptor and is therefore a preferred position for binding thereto moieties while not interrupting the CBD activity within the receptor.

The present inventor % have then devised several phospholipid-CBD conjugates, in which a phospholipid moiety is covalently attached either to position 6 of the CBD via a linker, or to position 5″ of the alkyl substituent at position 5′ (see, FIG. 1A). The rationale behind these conjugates is to attach to the CBD a moiety that would facilitate the CBD) loading by anchoring to the bilayer phospholipid membrane of the exosome.

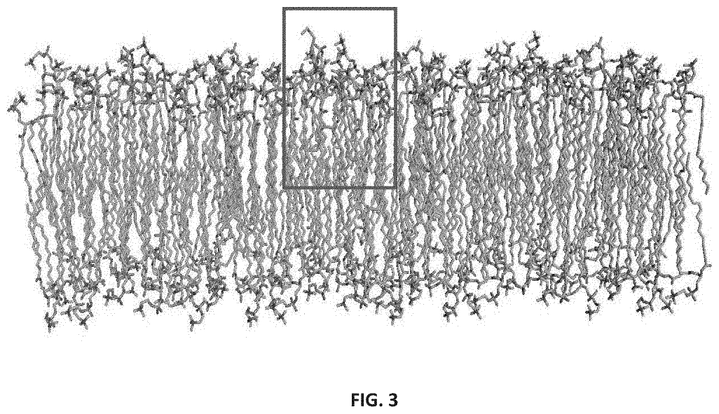

The CBD-phospholipid anchoring onto a bilayer phospholipid membrane was therefore computationally analyzed and the obtained configuration is shown in FIG. 3. As expected, CBD is presented outwards the membrane while the phospholipid is anchored to the membrane.

Exemplary designed CBD-phospholipid conjugates are presented in Table 1.

TABLE 1

| Compound | |
|---|---|
| No. | Structure |
| 1.1 | 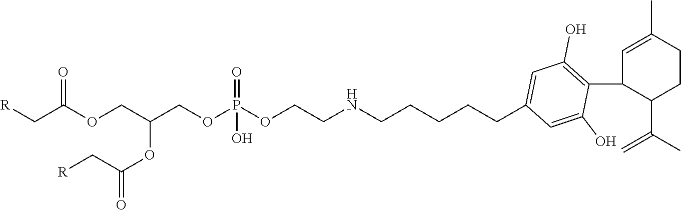 |
| 1.2 | 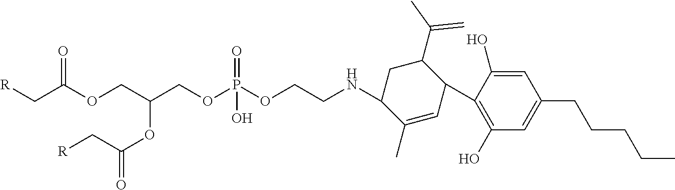 |
| 1.5 | 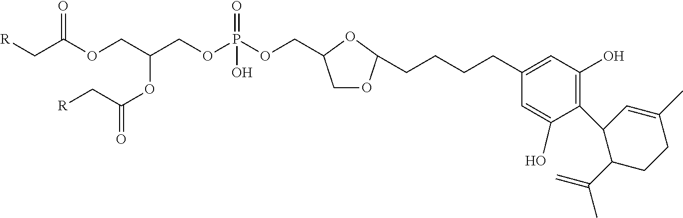 |
| 1.6 | 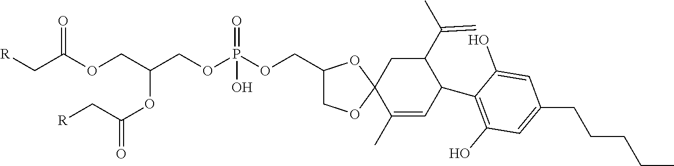 |
| PLC5 | 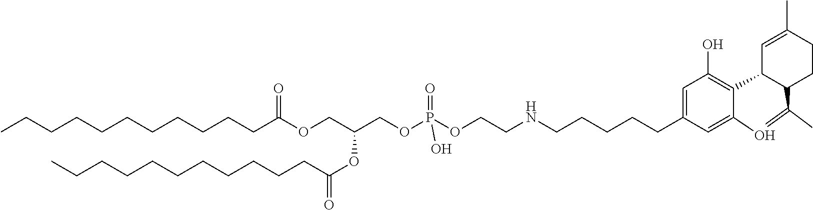 |
| PLC6 | 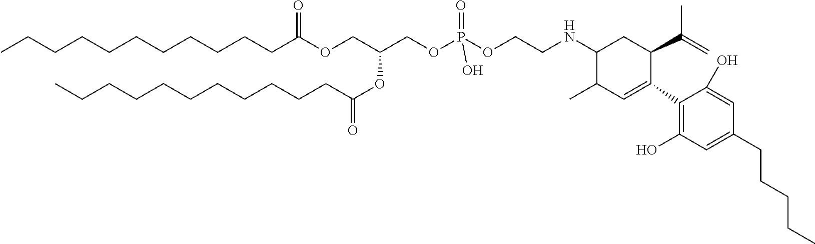 |

R is an alkyl of at least 4 carbon atoms in length as described herein for a fatty acyl.
PCL5 is an exemplary compound 1.1. PCL6 is an exemplary compound 1.2.

General Synthesis of Compounds 1.1:

Scheme 1 below presents a schematic depiction of the synthesis.

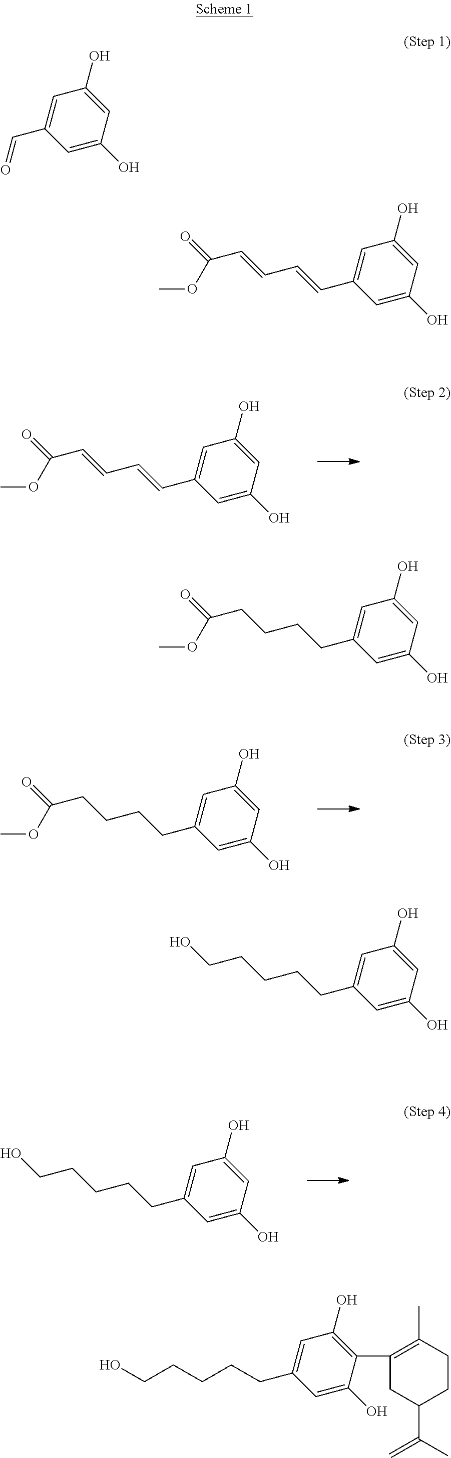

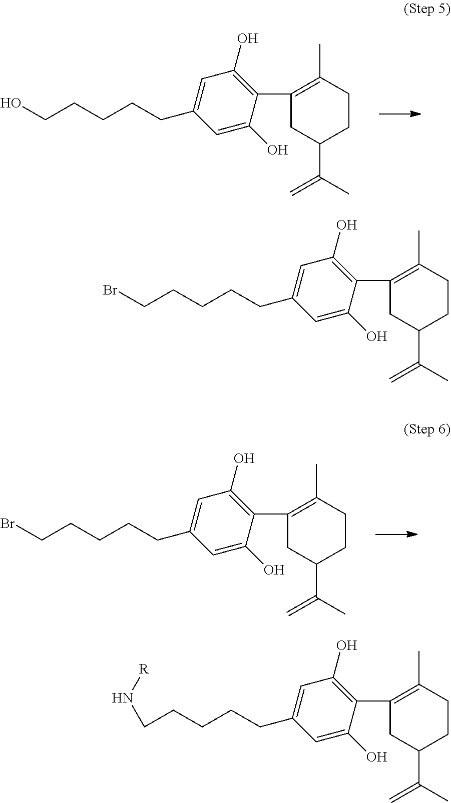

Step 1: The starting material 3,5-dihydroxybenzaldehyde (25 grams) and methyl crotonate (25 grams) are dissolved in 200 mL methanol. Sodium methoxide (5 grams) is added, and the mixture is refluxed overnight. The reaction is then quenched with 10 grams of acetic acid and 300 mL of ethyl acetate. The mixture is washed with 3×300 mL water. After evaporating the solvent, the mixture is purified by column chromatography on silica with hexane/ethyl acetate gradient as eluent, to thereby obtain the first intermediate product (e.g., 37 grams) in good yield.

Step 2: The intermediate product of step 1 (37 grams) is dissolved in 100 mL methanol. Pd/C (0.5 gram) was added, and the container is subjected to hydrogen atmosphere until the reaction is completed (about 5 hours). Ethyl acetate (300 mL) is then added and the mixture is washed with 3×300 mL water. The solvent is evaporated to thereby obtain the second intermediate product (e.g., 36 grams) in good yield.

Step 3: The second intermediate product (36 grams) is dissolved in 200 mL THF and lithium aluminum hydride (5 grams) is added carefully. After stirring overnight, the reaction is quenched with 10 mL acetic acid in 90 mL water. After filtration, the solvent is evaporated to thereby obtain the third intermediate product in good yield (e.g., 29 grams).

Step 4: The third intermediate product (26 grams) is dissolved in 250 mL DCM. Paramethandienol (15 grams) is added, and the resulting mixture is cooled in an ice bath. BF₃ etherate (1 mL) is added and the reaction is quenched after 45 minutes with 10 mL acetic acid in 90 mL water. The mixture is washed with 3×100 mL water. After evaporating the solvent, the mixture is purified by column chromatography on silica with hexane/ethyl acetate gradient as eluent to thereby obtain the fourth intermediate product (e.g., 10 grams).

Step 5: The fourth intermediate product (10 grams) is dissolved in 100 mL DCM. Phosphorus(III)bromide (5 grams) is then added and left to react overnight. The reaction is thereafter quenched with 10 grams potassium carbonate in 90 mL water. The mixture is washed with 2×100 mL water. After evaporating the solvent, the mixture is purified by column chromatography on silica with hexane/ethyl acetate gradient as eluent. The fifth intermediate product is obtained (e.g., 8 grams) in good yield.

Step 6: The fifth intermediate product (500 mg) is dissolved in 30 mL methanol and the obtained mixture is cooled in ice. The phospholipid (1 gram; denoted as R) is added and after 1 hour the reaction is completed. The mixture is washed with 2×100 mL water. After evaporating the solvent, the mixture is purified by column chromatography on silica with hexane/ethyl acetate gradient as eluent. The final product is obtained (1.1 grams) in good yield.

General Synthesis of Compounds 1.2:

Scheme 2 below presents a schematic depiction of the synthesis ( [symbol] represents [symbol] ).

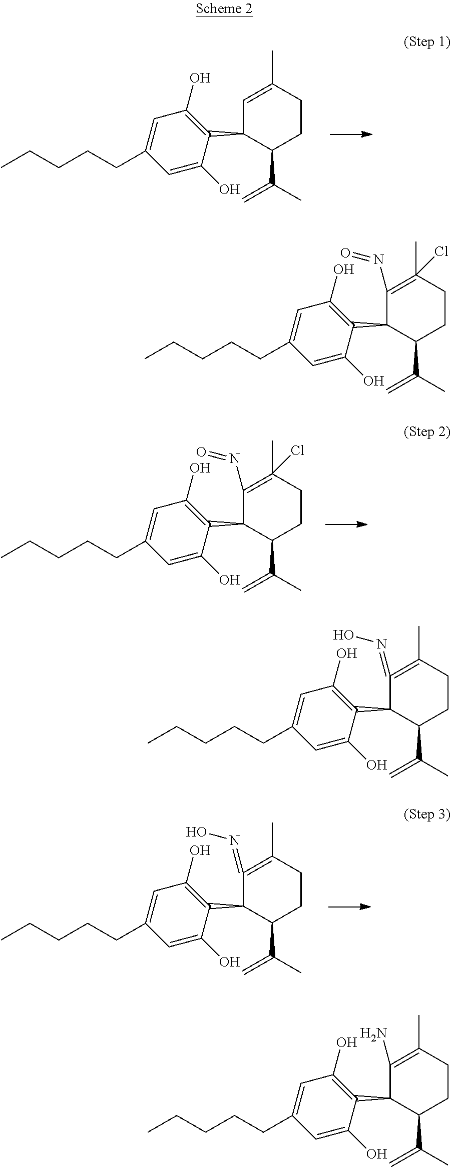

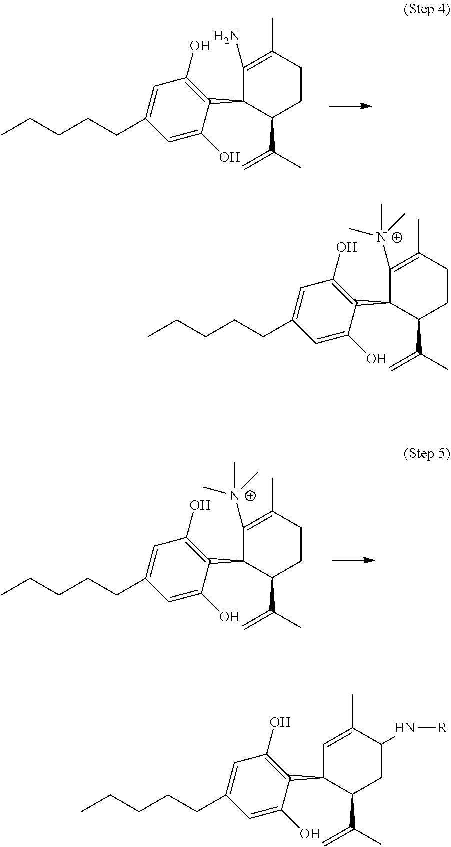

Step 1: The starting material (10 grams) is dissolved in 30 ml, ethanol. Nitrosyl chloride is bubbled into the solution until complete conversion. Ethyl acetate (50 grams) is added, and the mixture is washed with 2×100 mL water. After evaporating the solvent, the mixture is purified by column chromatography on silica with hexane % ethyl acetate gradient as eluent. The first intermediate product is obtained (e.g., S grams) in good yield.

Step 2: The first intermediate product (5 grams) is dissolved in 2 mL pyridine and 50 mL acetone. The solution is refluxed overnight. Ethyl acetate (50 grams) is then added, and the mixture is washed with 2×100 mL water. After evaporating the solvent, the mixture is purified by column chromatography on silica with hexane/ethyl acetate gradient as eluent. After further workup, the second intermediate product is obtained (e.g., 4 grams).

Step 3: The second intermediate product (4 grams) is dissolved in 30 mL ethanol, and the solution is cooled in an ice bath. Sodium borohydride (4 grams) is added carefully. After 1 hour, the solution is refluxed for an additional 1 hour. Ethyl acetate (50 grams) is then added, and the mixture is washed with 3×100 mL water. After evaporating the solvent, the mixture is purified by column chromatography on silica with hexane/ethyl acetate gradient as eluent. After further workup, the third intermediate product is obtained (e.g., 3 grains).

Step 4: The third intermediate product (3 grams) is dissolved in 20 mL methanol. Potassium carbonate (5 grams) and Methyl iodide (5 mL) are added. After stirring overnight, 20 mL of water are added. The fourth intermediate product (e.g., 4 grams) is crystalized out of this mixture.

Step 5: The fourth intermediate product (1 gram) is dissolved in 45 mL methanol. The phospholipid (1 gram, denoted as “R”) is added, and the mixture is stirred overnight. After evaporating the solvent, the mixture is purified by column chromatography on silica with hexane/ethyl acetate gradient as eluent to thereby obtain the final product (e.g., 1 gram).

### Example 5

### Loading CBD into Exosomes

A CBD or modified CBD as described herein is mixed with exosomes (e.g., MSC-derived exosomes) and the mixture is incubated at room temperature for about 1 hour.

In some of any of the embodiments described herein, loading CBD into exosomes or any other particles as described herein is effected such that a plurality of particles (e.g., in a range of from about 1×10¹⁰-1×10¹⁴) is mixed with a desired CBD dose, for example, in a range of from 1 to 50 mg/Kg/day.

In exemplary compositions according to the present embodiments, an amount of from about 1 mg to about 500 mg of CBD, or an equivalent amount of a modified CBD, including any intermediate values and subranges therebetween, is loaded into a plurality of particles (e.g., in a range of from about 1×10¹⁰-1×10¹⁴), to thereby prepare a composition as described herein.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A composition comprising a cell-derived particle and a cannabidiol associated with said particle.

2. The composition of claim 1, wherein said cell is a stem or progenitor cell.

3. The composition of claim 1, wherein said cell-derived particle is an exosome.

4. The composition of claim 1, wherein said cell-derived particle is a mesenchymal stem cell (MSC)-derived exosome.

5. The composition of claim 1, wherein the CBD is a modified CBD, which comprises at least one substituent or moiety attached to a position of the CBD, preferably to position 6 and/or 5″ of the CBD.

6. The composition of claim 5, wherein the modified CBD comprises a phospholipid moiety conjugated therewith, directly or via a linking moiety.

7. A method of treating a medical condition that can benefit from CBD and/or that is treatable by modulating an activity of a CB1 receptor in a subject in need thereof, the method comprising administering to the subject the composition of claim 1, wherein the medical condition is other than epilepsy.

8. The method of claim 7, wherein said cell is a stem or progenitor cell.

9. The method of claim 7, wherein said cell-derived particle is an exosome.

10. The method of claim 9 wherein said cell-derived particle is a mesenchymal stem cell (MSC)-derived exosome.

11. The method of claim 7, wherein said medical condition is selected from the group consisting of neurodegenarative disease, nerve injury, stroke, inflammation and infectious disease.

12. The method of claim 7, wherein said composition is administered intranasally or by inhalation.

13. The method of claim 7, wherein the CBD is a modified CBD, which comprises at least one substituent or moiety attached to a position of the CBD, preferably to position 6 and/or 5″ of the CBD.

14. The method of claim 13, wherein the modified CBD comprises a phospholipid moiety conjugated therewith, directly or via a linking moiety.

15. A method of treating a medical condition that can benefit from CBD and/or that is treatable by modulating an activity of a CB1 receptor in a subject in need thereof, the method comprising administering to the subject the composition of claim 1, wherein said cell is a stem or progenitor cell.

16. The method of claim 15, wherein said medical condition is selected from the group consisting of epilepsy, neurodegenerative disease, nerve injury, stroke, inflammation and infectious disease.

17. The method of claim 15, wherein said stem or progenitor cell is selected from the group consisting of a mesenchymal stem cell (MSC), neuronal stem cells (NSC), neuronal crest cell (NCC).

18. The method of claim 15 wherein said cell-derived particle is a mesenchymal stem cell (MSC)-derived exosome.

19. The method of claim 15, wherein said composition is administered intranasally or by inhalation.

20. The method of claim 15, wherein the CBD is a modified CBD, which comprises at least one substituent or moiety attached to a position of the CBD, preferably to position 6 and/or 5″ of the CBD.

21. The method of claim 15, wherein the modified CBD comprises a phospholipid moiety conjugated therewith, directly or via a linking moiety.
